(19) United States
(12) Patent Application Publication
(10) Pub. No.: US 2020/0223839 A1
(43) Pub. Date: Jul. 16, 2020

(54) FLUORO-CONTAINING COMPOUNDS, USE AND PREPARATION THEREOF

(71) Applicant: JIANGSU YINGKEBEITA YIYAOKEJI CO. LTD, Taizhou, Jiangsu (CN)

(72) Inventor: Yao MA, Lexington, MA (US)

(21) Appl. No.: 16/629,557

(22) PCT Filed: Jun. 27, 2018
(86) PCT No.: PCT/US18/39879

§ 371 Date: Jan. 8, 2020

Related U.S. Application Data
(60) Provisional application No. 62/530,263, filed on Jul. 9, 2017.

(51) Int. Cl. C07D 417/12

(52) U.S. Cl. CPC C07D 417/12

(57) ABSTRACT

The present teachings relate to a fluoro-containing compound, a composition thereof, a method of using the compound or the composition in treating a disease, and a method of preparing the compound. In a particular example, the compound is chosen from Formulae 4, 4f, 7, 8, 9, 10, 11, or 12, or a salt thereof, or a solvate of any of the foregoing.

This application claims the benefit of, and priority to, U.S. Patent Application Ser. No. 62/530,263, filed Jul. 9, 2017, the disclosure of which is incorporated herein by reference in its entirety.

The present teachings generally relate to fluoro-containing compounds, uses, and preparation thereof.

In one aspect, the present teachings relate to a compound of Formula I:

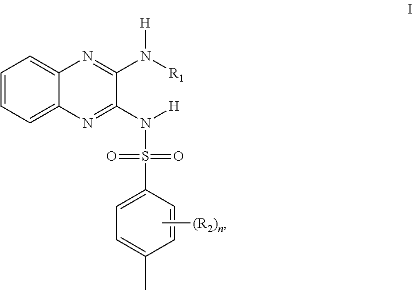

a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, the compound has Formula II:

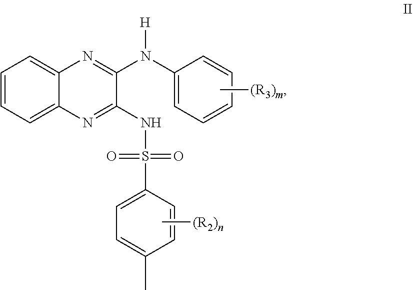

a salt thereof, or a solvate of any of the foregoing;
wherein R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carbocycle or heterocyclic;
m is 0, 1, 2, 3, or 4; and
n and R₂ are as defined herein.

In some embodiments, the compound has Formula IIa:

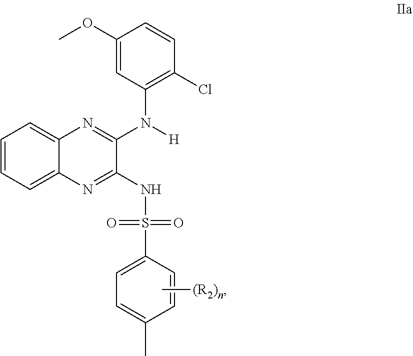

a salt thereof, or a solvate of any of the foregoing;
wherein n and R₂ are as defined herein.

In some embodiments, the compound has Formula III:

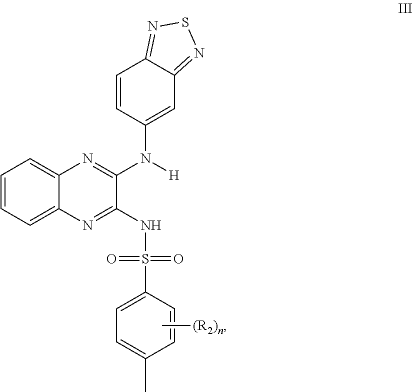

a salt thereof, or a solvate of any of the foregoing, wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, the present teachings relate to a compound of Formula IV:

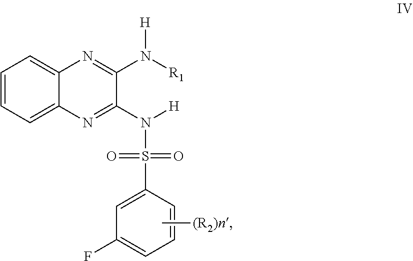

a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; n′ is 0, 1, 2, 3, or 4.

In some embodiments, the compound has Formula 4f:

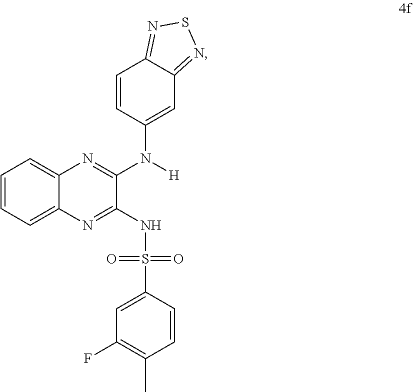 [SMILES: Cc1ccc(S(=O)(=O)Nc2nc3ccccc3nc2Nc2ccc3nsnc3c2)cc1F]

a salt thereof, or a solvate of any of the foregoing.

In one aspect, the present teachings relate to a composition comprising a compound of the present teachings. In some embodiments, the composition is a pharmaceutical composition.

In one aspect, the present teachings relate to a method of treating a subject in need thereof comprising administering to the subject a therapeutically effect amount of a compound according to the present teachings. In some embodiments, the method is a method of treating a disease in a subject. In some embodiments, the disease is a cancer. In some embodiments, the present teachings relate to a method of treating cancer in a subject comprising administering to a subject a therapeutically effect amount of the compound of the present teachings. In some embodiments, the present teachings relate to a method of treating cancer in a subject comprising administering to a subject a therapeutically effect amount of the composition of the present teachings.

### BRIEF DESCRIPTION OF DRAWINGS

The following drawings form part of the present teachings and are included to demonstrate certain aspects of the present teachings. The present teachings may be better understood by reference to one or more of these drawings in combination with the detailed description of specific embodiments presented herein.

FIG. 1 illustrates the results of an exemplary cytotoxicity assay of a compound of the present teachings as well as certain comparator compounds to human breast cancer line MDA-MB-468 according to some embodiments of the present teachings.

FIG. 2 illustrates the results (tumor sizes) of an exemplary in vivo xenograft study a compound of the present teachings as well as certain comparator compounds to human breast cancer line MDA-MB-468 implanted in mice according to some embodiments of the present teachings.

FIG. 3 illustrates the results (mice weights) of an exemplary in vivo xenograft study a compound of the present teachings as well as certain comparator compounds to human breast cancer line MDA-MB-468 implanted in mice according to some embodiments of the present teachings.

FIG. 4 illustrates the results (tumor sizes) of another exemplary in vivo xenograft study a compound of the present teachings as well as certain comparator compounds to human breast cancer line MDA-MB-468 implanted in mice according to some embodiments of the present teachings.

FIG. 5 illustrates the results (mice weights) of another exemplary in vivo xenograft study a compound of the present teachings as well as certain comparator compounds to human breast cancer line MDA-MB-468 implanted in mice according to some embodiments of the present teachings.

FIG. 6 illustrates the results (tumor sizes) of an exemplary in vivo xenograft study a compound of the present teachings as well as certain comparator compounds to human breast cancer line MDA-MB-453 implanted in mice according to some embodiments of the present teachings.

FIG. 7 illustrates the results (mice weights) of an exemplary in vivo xenograft study a compound of the present teachings as well as certain comparator compounds to human breast cancer line MDA-MB-453 implanted in mice according to some embodiments of the present teachings.

FIG. 8 illustrates the results (tumor sizes) of an exemplary in vivo xenograft study a compound of the present teachings as well as certain comparator compounds to human liver cancer line Hep3B implanted in mice according to some embodiments of the present teachings.

FIG. 9 illustrates the results (mice weights) of an exemplary in vivo xenograft study a compound of the present teachings as well as certain comparator compounds to human liver cancer line Hep3B implanted in mice according to some embodiments of the present teachings.

In the present teachings, where an element or component is said to be included in and/or selected from a list of recited elements or components, it should be understood that the element or component can be any one of the recited elements or components, or can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “a,” “an,” and “the” and similar references in the context of the present teachings (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the teachings and does not pose a limitation on the scope of the claims. No language in the present teachings should be construed as indicating any non-claimed element as essential to the practice of the present teachings.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined herein. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of the terms “comprise,” “comprises,” “comprising,” “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, +5%, +2%, or +1% variation from the nominal value unless otherwise indicated or inferred.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₁-C₆), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

It should be understood that the order of steps or order for performing certain actions is immaterial so long as the present teachings remain operable. Moreover, two or more steps or actions may be conducted simultaneously.

As used herein, the terms “therapeutic agent” and “therapeutic agents” refer to any agent(s) which can be used to furnish pharmacological activity or other direct effect in the diagnosis, cure, mitigation, treatment, or prevention of disease or to affect the structure or any function of the human body. In certain embodiments, the term “therapeutic agent” includes a compound provided herein. In certain embodiments, a therapeutic agent is an agent which is known to be useful for, or has been or is currently being used for the treatment or prevention of a disorder or one or more symptoms thereof.

The term “small molecule”, as used herein, generally refers to an organic molecule that is less than 2000 g/mol in molecular weight, less than 1500 g/mol, less than 1000 g/mol, less than 800 g/mol, or less than 500 g/mol.

The term “molecular weight”, as used herein, generally refers to the mass or average mass of a molecule of a material.

A dash (“-”) that is not between two letters or symbols is used to indicate a point of attachment for a substituent. For example, —CONH₂ is attached through the carbon atom (C).

The term “alkane” refers to a saturated aliphatic hydrocarbon compound, including a straight-chain alkane, branched-chain alkane, cycloalkane, alkyl-substituted cycloalkane, and cycloalkyl-substituted alkane that has 30 or fewer carbon atoms in its backbone (e.g., C₁-C₃₀ for straight chains, C₃-C₃₀ for branched chains). In some embodiments, the alkane has 20 or fewer, 12 or fewer, or 7 or fewer carbon atoms in its backbone.

The terms “alkene” and “alkyne” refer to unsaturated aliphatic analogous in length and possible substitution to the alkane described above, but that contain at least one double or triple bond respectively.

The term “aromatic” refers to C₅-C₂₀-membered aromatic, heterocyclic, fused aromatic, fused heterocyclic, biaromatic, or bihetereocyclic compound.

The term “alkyl” refers to the radical of alkanes, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments, cycloalkyls have from 3-10 carbon atoms in their ring structure, e.g. have 5, 6, or 7 carbons in the ring structure. The term “alkyl” (or “lower alkyl”) as used throughout the present teachings, examples, and claims is intended to include both “unsubstituted alkyls” and “substituted alkyls,” the latter of which refers to alkyl moieties having one or more substituents, such as those described herein, replacing one or more hydrogens on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxy, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, aromatic, or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the present teachings, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate, with one or more substituents, each of which is discussed herein. For instance, the substituents of a substituted alkyl may include halogen, hydroxyl, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, 0, N, Si, P, Se, B, and S, wherein the phosphorous and sulfur atoms are optionally oxidized, and the nitrogen heteroatom is optionally quaternized. Heteroalkyls can be substituted with one or more substituents, each of which is discussed herein.

The term “alkylthio” refers to an alkyl group, as defined above, having a sulfur radical attached thereto. In some embodiments, the “alkylthio” moiety is represented by one of —S— alkyl, —S-alkenyl, and —S-alkynyl. Representative alkylthio groups include methylthio and ethylthio. The term “alkylthio” also encompasses cycloalkyl groups, alkenyl and cycloalkenyl groups, and alkyne groups. “Arylthio” refers to aryl or heteroaryl groups. Alkylthio groups can be substituted with one or more substituents, each of which is discussed herein.

The terms “alkenyl” and “alkynyl”, refer to unsaturated aliphatic groups analogous in length and possible substitution to the alkyls described above, but that contain at least one double or triple bond respectively.

The term “alkoxyl” or “alkoxy” as used herein refers to an alkyl group, as defined above, having an oxygen radical attached thereto. Representative alkoxy groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxy, such as can be represented by one of —O— alkyl, —O-alkenyl, and —O-alkynyl. Aroxy can be represented by —O-aryl or O-heteroaryl, wherein aryl and heteroaryl are as defined below. The alkoxy and aroxy groups can be substituted as described with one or more substituents, each of which is discussed herein.

The terms “amine” and “amino” are art-recognized and refer to both unsubstituted and substituted amines, e.g., a moiety that can be represented by the general formula:

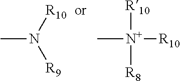

wherein R₉, R₁₀, and R′₁₀ each independently represent a hydrogen, an alkyl, an alkenyl, —(CH₂)_{m′}—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m′ is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (with one or more substituents, each of which is discussed herein) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

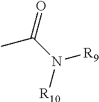

wherein R₉ and R₁₀ are as defined herein.

“Aryl,” as used herein, refers to C₅-C₁₀-membered aromatic, heterocyclic, fused aromatic, fused heterocyclic, biaromatic, or bihetereocyclic ring systems. Broadly defined, “aryl,” as used herein, includes 5-, 6-, 7-, 8-, 9-, and 10-membered single-ring aromatic groups that may include from zero to four heteroatoms, for example, benzene, pyrrole, furan, thiophene, imidazole, oxazole, triazole, triazole, pyrazole, pyridine, pyrazine, pyridazine and pyrimidine, and the like. Those aryl groups having heteroatoms in the ring structure may also be referred to as “aryl heterocycles,” “heteroaromatics,” or “heteroaryl.” The aromatic ring can be substituted at one or more ring positions with one or more substituents with one or more substituents, each of which is discussed herein. For example, the one or more substituents can include, but not limited to, halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzothiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3b]tetrahydrofuranyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or nonaromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀)alkyl, phenyl or benzyl, and optionally containing 1-3 double bonds and optionally substituted with one or more substituents, each of which is discussed herein. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl. Heterocyclic groups can optionally be substituted at one or more positions with one or more substituents, each of which is discussed herein, including, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, and —CN.

The term “carbonyl” is art-recognized and includes such moieties as can be represented by the general formula:

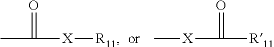

wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester.” Where X is an oxygen and R₁₁ is as defined above, the moiety is referred to herein as a carboxyl group, and particularly when R₁₁ is a hydrogen, the formula represents a “carboxylic acid.” Where X is an oxygen and R′₁₁ is hydrogen, the formula represents a “formate.”

In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Examples of heteroatoms are boron, nitrogen, oxygen, phosphorus, sulfur, and selenium. Other heteroatoms include silicon and arsenic.

As used herein, the term “nitro” means —NO₂; the term “halogen” designates —F, —Cl, —Br, or —I; the term “sulfhydryl” means —SH; the term “hydroxyl” means —OH; and the term “sulfonyl” means —SO₂—.

As used herein, “fluorinated” refers to chemical compounds each having at least one carbon-bonded hydrogen replaced by a fluorine (—F), and can include perfluorinated compounds.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

In various embodiments, the substituent is alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, or thioketone, each of which optionally is substituted with one or more suitable substituents. In some embodiments, the substituent is alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, or thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxy, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

The term “subject” generally refers to an organism to which a compound or pharmaceutical composition described herein can be administered. A subject can be a mammal or mammalian cell, including a human or human cell. The term also refers to an organism, which includes a cell or a donor or recipient of such cell. In various embodiments, the term “subject” refers to any animal (e.g., a mammal), including, but not limited to humans, mammals and non-mammals, such as non-human primates, mice, rabbits, sheep, dogs, cats, horses, cows, chickens, amphibians, and reptiles, which is to be the recipient of a compound or pharmaceutical composition described herein. Under some circumstances, the terms “subject” and “patient” are used interchangeably herein in reference to a human subject.

The terms “effective amount” and “therapeutically effective amount” refer to that amount of a compound or pharmaceutical composition described herein that is sufficient to effect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment,” “treating,” “ameliorating,” and “encouraging” may be used interchangeably herein. These terms refer to an approach for obtaining beneficial or desired results including, but not limited to, therapeutic benefit and/or prophylactic benefit. By therapeutic benefit it is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical composition may be administered to a patient at risk of developing a particular disease, or to a patient reporting one or more of the physiological symptoms of a disease, even though a diagnosis of this disease may not have been made.

The term “cancer” in a subject refers to the presence of cells possessing characteristics typical of cancer-causing cells, such as uncontrolled proliferation, immortality, metastatic potential, rapid growth and proliferation rate, and/or certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, colon cancer, rectal cancer, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

In some embodiments, the cancer is chosen from colon adenocarcinoma, rectal adenocarcinoma, gastric adenocarcinoma, gastroesophageal junction adenocarcinoma, esophageal adenocarcinoma, hepatocellular carcinoma, ovarian cancer, platinum-resistant ovarian cancer, pancreatic adenocarcinoma, breast cancer, triple negative breast cancer, ovarian cancer, cholangiocarcinoma, melanoma, small cell lung cancer, and non-small cell lung cancer. In some embodiments, the cancer is colorectal cancer. In some embodiments, the cancer is a lung cancer. In some embodiments, the cancer is small cell lung cancer. In some embodiments, the cancer is non-small cell lung cancer. In some embodiments, the cancer is breast cancer.

In one aspect, the present teachings relate to a compound of Formula I:

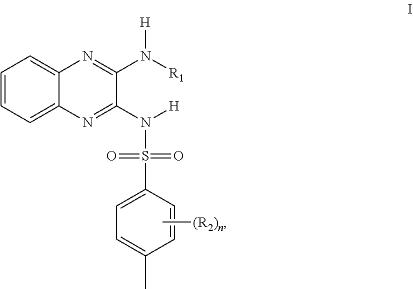

a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, R₁ is a 5-, 6-, 7-, 8-, 9-, or 10-membered single-ring aromatic group, wherein each of the 5-, 6-, 7-, 8-, 9-, and 10-membered single-ring aromatic groups includes 0, 1, 2, 3, or 4 heteroatom and is optionally substituted with 1, 2, 3, or 4 substituents.

In some embodiments, R₁ is a 6- or 10-membered single-ring aromatic group, wherein each of the 6- and 10-membered single-ring aromatic groups is optionally substituted with 1, 2, 3, or 4 substituents.

In some embodiments, R₁ is phenyl optionally substituted with 1, 2, 3, or 4 substituents. In some embodiments, the compound has Formula II:

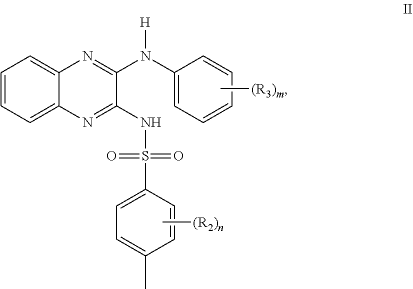

a salt thereof, or a solvate of any of the foregoing;
wherein R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic; and
m is 0, 1, 2, 3, or 4.

In some embodiments, m is 2 and R₃ at each occurrence independently is chosen from halogen, alkyl, hydroxyl, alkoxy, amino, nitro, carbonyl, alkylthio, sulfonyl, —CF₃, or —CN. In some embodiments, m is 2 and R₃ at each occurrence independently is chosen from halogen or alkoxy. In some embodiments, the compound has Formula IIa:

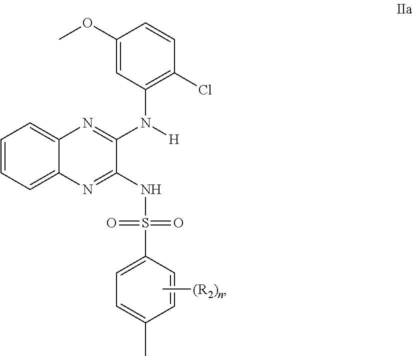

a salt thereof, or a solvate of any of the foregoing.

In some embodiments, R₁ is a 5-, 6-, 7-, 8-, 9-, or 10-membered aromatic group, wherein each of the 5-, 6-, 7-, 8-, 9-, and 10-membered aromatic groups includes 1, 2, 3, or 4 heteroatoms and is optionally substituted with 1, 2, 3, or 4 substituents. In some embodiments, R₁ is a heteroaryl optionally substituted with 1, 2, 3, or 4 substituents. In some embodiments, R₁ is chosen from benzimidazolyl, benzofuranyl, benzothiofuranyl, benzoxazolyl, benzothiazolyl, benzothiadiazolyl, benzotriazolyl, benzisoxazolyl, benzisothiazolyl, carbazolyl, cinnolinyl, furanyl, furazanyl, imidazolyl, indazolyl, indolyl, isobenzofuranyl, isochromanyl, isoindazolyl, isoindolyl, isoquinolinyl, isothiazolyl, isoxazolyl, morpholinyl, naphthyridinyl, oxadiazolyl, pyrimidinyl, pteridinyl, purinyl, pyranyl, pyrazinyl, pyrazolyl, pyridazinyl, pyridyl, pyrimidinyl, pyrrolyl, quinazolinyl, quinolinyl, quinoxalinyl, tetrazolyl, thiadiazolyl, thianthrenyl, thienyl, or xanthenyl and each of benzimidazolyl, benzofuranyl, benzothiofuranyl, benzoxazolyl, benzothiazolyl, benzothiadiazolyl, benzotriazolyl, benzisoxazolyl, benzisothiazolyl, carbazolyl, cinnolinyl, furanyl, furazanyl, imidazolyl, indazolyl, indolyl, isobenzofuranyl, isochromanyl, isoindazolyl, isoindolyl, isoquinolinyl, isothiazolyl, isoxazolyl, morpholinyl, naphthyridinyl, oxadiazolyl, pyrimidinyl, pteridinyl, purinyl, pyranyl, pyrazinyl, pyrazolyl, pyridazinyl, pyridyl, pyrimidinyl, pyrrolyl, quinazolinyl, quinolinyl, quinoxalinyl, tetrazolyl, thiadiazolyl, thianthrenyl, thienyl, and xanthenyl is optionally substituted with 1, 2, 3, or 4 substituents. In some embodiments, R₁ is chosen from benzimidazolyl, benzofuranyl, benzothiofuranyl, benzoxazolyl, benzothiazolyl, benzothiadiazolyl, benzotriazolyl, benzisoxazolyl, or benzisothiazolyl and each of benzimidazolyl, benzofuranyl, benzothiofuranyl, benzoxazolyl, benzothiazolyl, benzothiadiazolyl, benzotriazolyl, benzisoxazolyl, and benzisothiazolyl is optionally substituted with 1, 2, 3, or 4 substituents.

In some embodiments, the compound has Formula III:

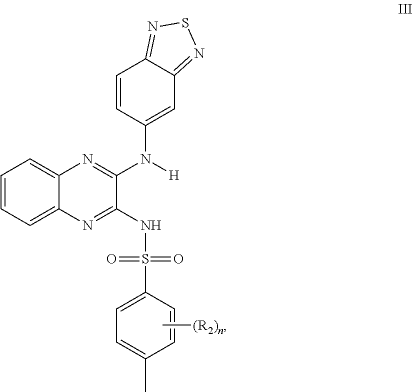

a salt thereof, or a solvate of any of the foregoing, wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, n is 1, 2, or 3. In some embodiments, n is 1.

In some embodiments, R₂ at each occurrence independently is chosen from halogen, hydroxyl, alkoxy, amino, nitro, —CF₃, or —CN. In some embodiments, R₂ at each occurrence independently is chosen from halogen. In some embodiments, R₂ at each occurrence independently is F.

In some embodiments, the present teachings relate to a compound of Formula IV:

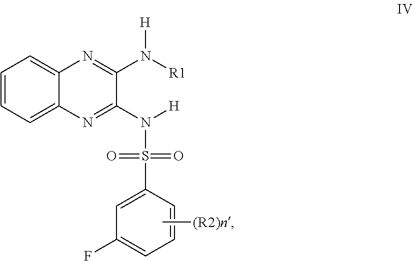

a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
n′ is 0, 1, 2, 3, or 4.

In some embodiments, n′ is 0, 1, or 2. In some embodiments, n′ is 0. In some embodiments, n′ is 1. In some embodiments, n′ is 2.

In some embodiments, the present teachings relate to a compound of Formula V:

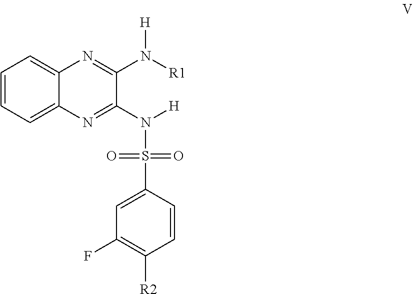

a salt thereof, or a solvate of any of the foregoing; wherein
R₁ is an aryl group; and
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, R₁ is a 5-, 6-, 7-, 8-, 9-, or 10-membered single-ring aromatic group, wherein each of the 5-, 6-, 7-, 8-, 9-, and 10-membered single-ring aromatic groups includes 0, 1, 2, 3, or 4 heteroatom and is optionally substituted with 1, 2, 3, or 4 substituents.

In some embodiments, R₁ is a 6- or 10-membered single-ring aromatic group, wherein each of the 6- and 10-membered single-ring aromatic groups is optionally substituted with 1, 2, 3, or 4 substituents. In some embodiments, R₁ is phenyl optionally substituted with 1, 2, 3, or 4 substituents.

In some embodiments, the present teachings relate to a compound of Formula VI:

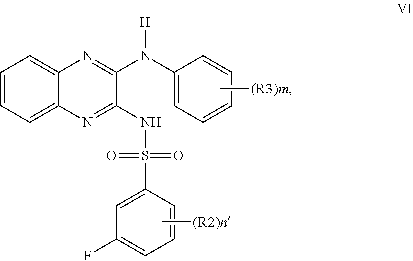

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic;
m is 0, 1, 2, 3, or 4; and
n′ is 0, 1, 2, 3, or 4.

some embodiments, n′ is 0, 1, or 2. In some embodiments, n′ is 0. In some embodiments, n′ is 1. In some embodiments, n′ is 2.

In some embodiments, the present teachings relate to a compound of Formula VII:

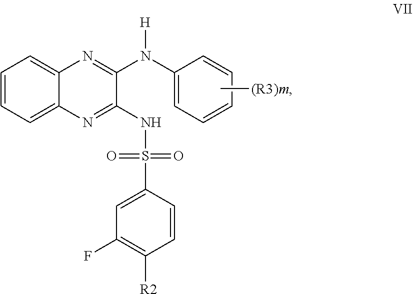

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic; and
m is 0, 1, 2, 3, or 4.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, R₁ is a 5-, 6-, 7-, 8-, 9-, or 10-membered aromatic group, wherein each of the 5-, 6-, 7-, 8-, 9-, and 10-membered aromatic groups includes 1, 2, 3, or 4 heteroatoms and is optionally substituted with 1, 2, 3, or 4 substituents. In some embodiments, R₁ is a heteroaryl optionally substituted with 1, 2, 3, or 4 substituents. In some embodiments, R₁ is chosen from benzimidazolyl, benzofuranyl, benzothiofuranyl, benzoxazolyl, benzothiazolyl, benzothiadiazolyl, benzotriazolyl, benzisoxazolyl, benzisothiazolyl, carbazolyl, cinnolinyl, furanyl, furazanyl, imidazolyl, indazolyl, indolyl, isobenzofuranyl, isochromanyl, isoindazolyl, isoindolyl, isoquinolinyl, isothiazolyl, isoxazolyl, morpholinyl, naphthyridinyl, oxadiazolyl, pyrimidinyl, pteridinyl, purinyl, pyranyl, pyrazinyl, pyrazolyl, pyridazinyl, pyridyl, pyrimidinyl, pyrrolyl, quinazolinyl, quinolinyl, quinoxalinyl, tetrazolyl, thiadiazolyl, thianthrenyl, thienyl, or xanthenyl and each of benzimidazolyl, benzofuranyl, benzothiofuranyl, benzoxazolyl, benzothiazolyl, benzothiadiazolyl, benzotriazolyl, benzisoxazolyl, benzisothiazolyl, carbazolyl, cinnolinyl, furanyl, furazanyl, imidazolyl, indazolyl, indolyl, isobenzofuranyl, isochromanyl, isoindazolyl, isoindolyl, isoquinolinyl, isothiazolyl, isoxazolyl, morpholinyl, naphthyridinyl, oxadiazolyl, pyrimidinyl, pteridinyl, purinyl, pyranyl, pyrazinyl, pyrazolyl, pyridazinyl, pyridyl, pyrimidinyl, pyrrolyl, quinazolinyl, quinolinyl, quinoxalinyl, tetrazolyl, thiadiazolyl, thianthrenyl, thienyl, and xanthenyl is optionally substituted with 1, 2, 3, or 4 substituents. In some embodiments, R₁ is chosen from benzimidazolyl, benzofuranyl, benzothiofuranyl, benzoxazolyl, benzothiazolyl, benzothiadiazolyl, benzotriazolyl, benzisoxazolyl, or benzisothiazolyl and each of benzimidazolyl, benzofuranyl, benzothiofuranyl, benzoxazolyl, benzothiazolyl, benzothiadiazolyl, benzotriazolyl, benzisoxazolyl, and benzisothiazolyl is optionally substituted with 1, 2, 3, or 4 substituents.

In some embodiments, the present teachings relate to a compound of Formula VIII:

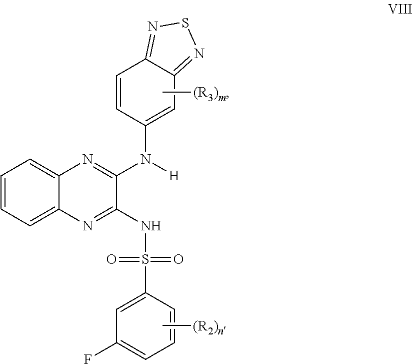

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
m is 0, 1, 2, 3, or 4; and
n′ is 0, 1, 2, 3, or 4.

In some embodiments, m is 0, 1, 2, or 3. In some embodiments, m is 0. In some embodiments, m is 1. In some embodiments, m is 2.

In some embodiments, R₃ at each occurrence independently is chosen from halogen, azide, alkyl, aralkyl, alkenyl, alkynyl, cycloalkyl, hydroxyl, alkoxy, amino, nitro, carbonyl, carboxyl, silyl, ether, alkylthio, sulfonyl, sulfonamido, ketone, aldehyde, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, nitro, sulfonyl, sulfonamido, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, ester, heterocyclyl, aromatic or heteroaromatic moieties, —CF₃, or —CN.

In some embodiments, n′ is 0, 1, or 2. In some embodiments, n′ is 0. In some embodiments, n′ is 1. In some embodiments, n′ is 2.

In some embodiments, the present teachings relate to a compound of Formula IX:

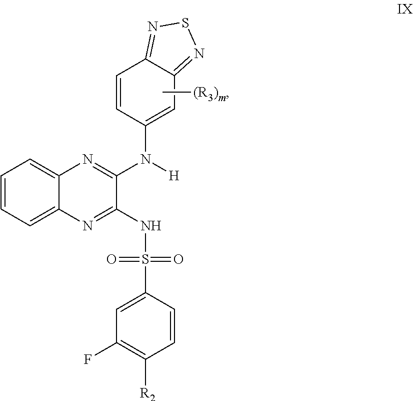

a salt thereof, or a solvate of any of the foregoing;
wherein
R₂ is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
m is 0, 1, 2, 3, or 4.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, m is 0, 1, 2, or 3. In some embodiments, m is 0. In some embodiments, m is 1. In some embodiments, m is 2.

In some embodiments, R₃ at each occurrence independently is chosen from halogen, azide, alkyl, aralkyl, alkenyl, alkynyl, cycloalkyl, hydroxyl, alkoxy, amino, nitro, carbonyl, carboxyl, silyl, ether, alkylthio, sulfonyl, sulfonamido, ketone, aldehyde, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, nitro, sulfonyl, sulfonamido, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, ester, heterocyclyl, aromatic or heteroaromatic moieties, —CF₃, or —CN.

In some embodiments, the compound is chosen from:

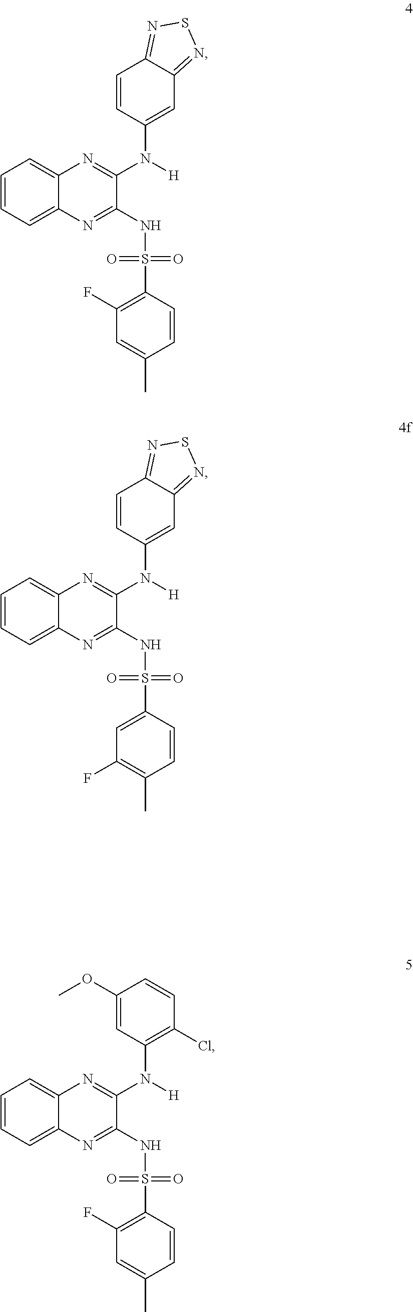 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F ; COc1ccc(Cl)c(Nc2nc3ccccc3nc2NS(=O)(=O)c2ccc(C)cc2F)c1]

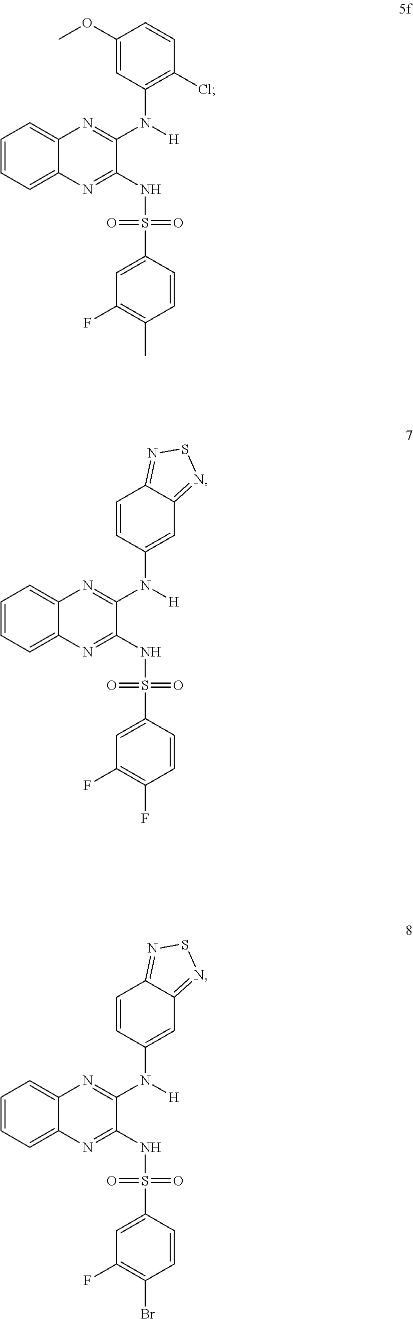 [SMILES: COc1ccc(Cl)c(Nc2nc3ccccc3nc2NS(=O)(=O)c2ccc(C)c(F)c2)c1 ; O=S(=O)(Nc1nc2ccccc2nc1Nc1ccc2nsnc2c1)c1ccc(F)c(F)c1 ; O=S(=O)(Nc1nc2ccccc2nc1Nc1ccc2nsnc2c1)c1ccc(Br)c(F)c1]

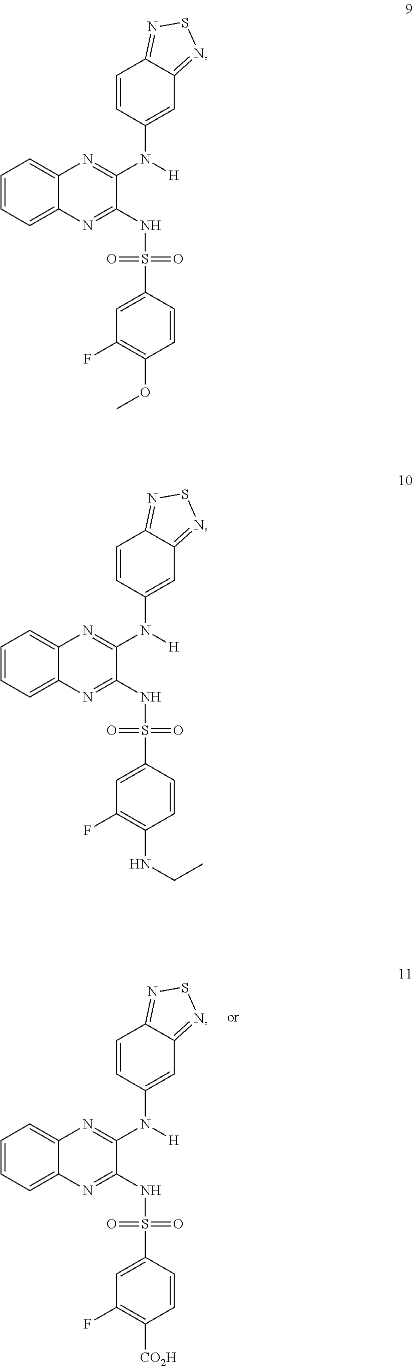 [SMILES: COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F ; O=C(O)c1ccc(S(=O)(=O)Nc2nc3ccccc3nc2Nc2ccc3nsnc3c2)cc1F ; "or"]

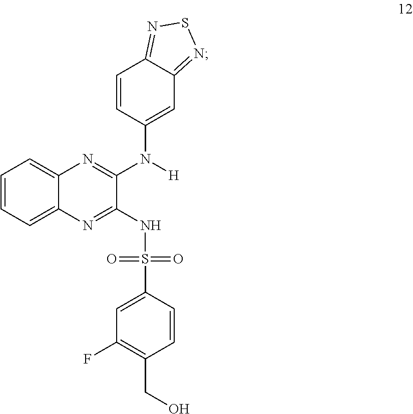 [SMILES: O=S(=O)(Nc1nc2ccccc2nc1Nc1ccc2nsnc2c1)c1ccc(CO)c(F)c1]

a salt thereof, or a solvate of any of the foregoing.

In some embodiments, the compound is chosen from:

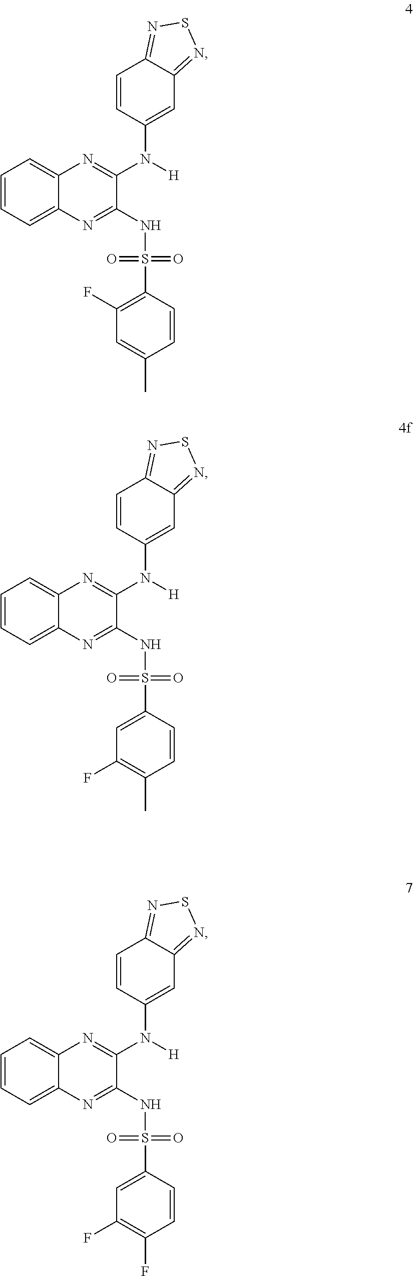 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F ; O=S(=O)(Nc1nc2ccccc2nc1Nc1ccc2nsnc2c1)c1ccc(F)c(F)c1]

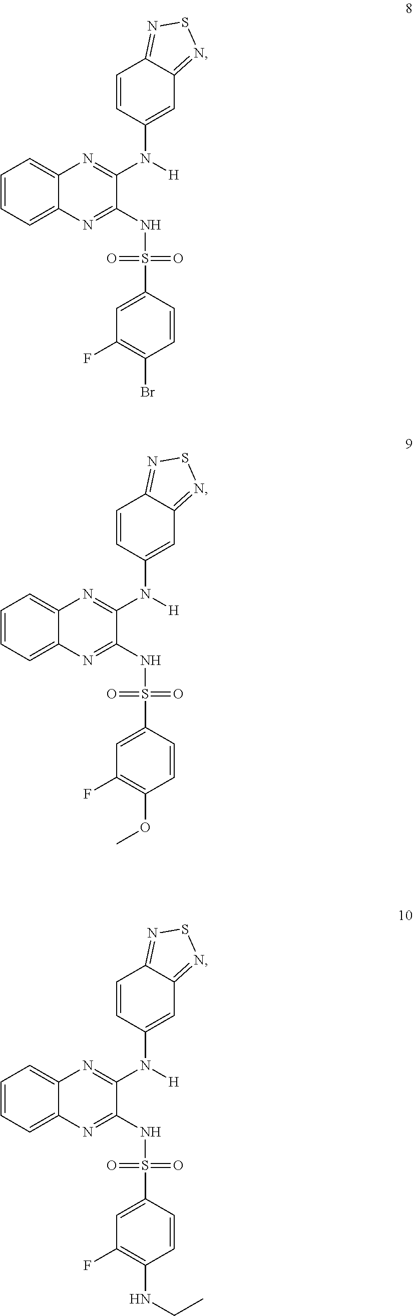 [SMILES: O=S(=O)(Nc1nc2ccccc2nc1Nc1ccc2nsnc2c1)c1ccc(Br)c(F)c1 ; COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F]

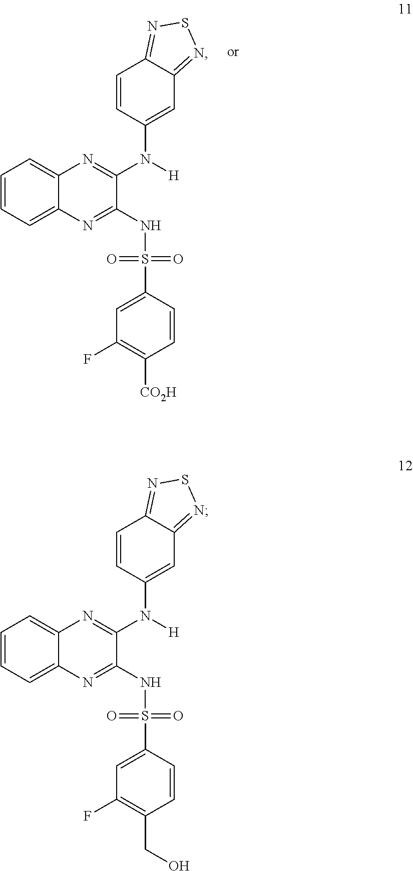 [SMILES: O=C(O)c1ccc(S(=O)(=O)Nc2nc3ccccc3nc2Nc2ccc3nsnc3c2)cc1F ; "or" ; O=S(=O)(Nc1nc2ccccc2nc1Nc1ccc2nsnc2c1)c1ccc(CO)c(F)c1]

a salt thereof, or a solvate of any of the foregoing.

In some embodiments, the compound has Formula 4f:

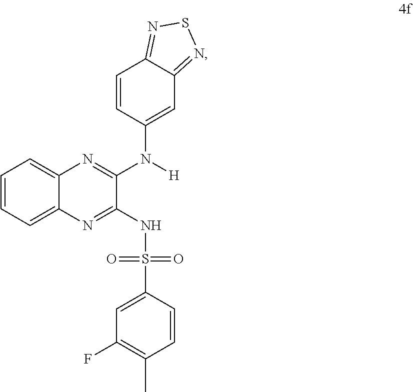 [SMILES: Cc1ccc(S(=O)(=O)Nc2nc3ccccc3nc2Nc2ccc3nsnc3c2)cc1F]

a salt thereof, or a solvate of any of the foregoing.

In some embodiments, the compound has Formula 7:

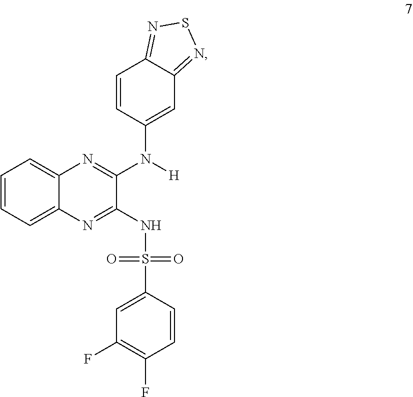 [SMILES: O=S(=O)(Nc1nc2ccccc2nc1Nc1ccc2nsnc2c1)c1ccc(F)c(F)c1]

a salt thereof, or a solvate of any of the foregoing.

In one aspect, the present teachings relate to at least one compound disclosed herein in the form of a composition. In some embodiments, the compositions may comprise the at least one compound of Formula I, II, IIa, III, IV, V, VI, VII, VIII, IX, 4, 4f, 5, 5f, 7, 8, 9, 10, 11, or 12, and at least one pharmaceutically acceptable carrier. In some embodiments, the composition includes a compound of Formula I and at least one pharmaceutically acceptable carrier. In some embodiments, the composition includes a compound of Formula II and at least one pharmaceutically acceptable carrier. In some embodiments, the composition includes a compound of Formula IIa and at least one pharmaceutically acceptable carrier. In some embodiments, the composition includes a compound of Formula III and at least one pharmaceutically acceptable carrier. In some embodiments, the composition includes a compound of Formula IV and at least one pharmaceutically acceptable carrier. In some embodiments, the composition includes a compound of Formula V and at least one pharmaceutically acceptable carrier. In some embodiments, the composition includes a compound of Formula VI and at least one pharmaceutically acceptable carrier. In some embodiments, the composition includes a compound of Formula VII and at least one pharmaceutically acceptable carrier. In some embodiments, the composition includes a compound of Formula VIII and at least one pharmaceutically acceptable carrier. In some embodiments, the composition includes a compound of Formula IX and at least one pharmaceutically acceptable carrier. In some embodiments, the composition includes a compound of at least one compound of Formula 4, 4f, 5, 5f, 7, 8, 9, 10, 11, or 12, and at least one pharmaceutically acceptable carrier. In some embodiments, the composition includes a compound of Formula 4f and at least one pharmaceutically acceptable carrier. In some embodiments, the composition includes a compound of Formula 7 and at least one pharmaceutically acceptable carrier.

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions. In some embodiments, the composition is a pharmaceutical composition.

In one aspect, the present teachings relate to a method of treating a subject in need thereof comprising administering to the subject a therapeutically effect amount of at least one compound disclosed in the present teachings. In some embodiments, the method is a method of treating a disease in a subject. In some embodiments, the disease is a cancer. In some embodiments, the present teachings relate to a method of treating cancer in a subject comprising administering to a subject a therapeutically effect amount of at least one compound of the present teachings. In some embodiments, the present teachings relate to a method of treating cancer in a subject comprising administering to a subject a therapeutically effect amount of the composition of the present teachings.

In some embodiments, the present teachings relate to a method of treating a subject in need thereof comprising administering to the subject a therapeutically effect amount of a compound of Formula I:

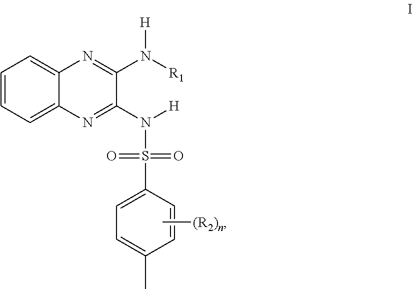

a salt thereof, or a solvate of any of the foregoing; wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, R₁ is a 5-, 6-, 7-, 8-, 9-, or 10-membered single-ring aromatic group, wherein each of the 5-, 6-, 7-, 8-, 9-, and 10-membered single-ring aromatic groups includes 0, 1, 2, 3, or 4 heteroatom and is optionally substituted with 1, 2, 3, or 4 substituents.

In some embodiments, R₁ is a 6- or 10-membered single-ring aromatic group, wherein each of the 6- and 10-membered single-ring aromatic groups is optionally substituted with 1, 2, 3, or 4 substituents.

In some embodiments, R₁ is phenyl optionally substituted with 1, 2, 3, or 4 substituents.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula II:

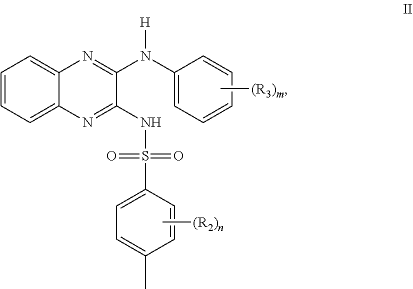

a salt thereof, or a solvate of any of the foregoing;
wherein
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic;
m is 0, 1, 2, 3, or 4; and
n and R₂ are as defined herein.

In some embodiments, the present teachings relate to a method of treating a subject in need thereof comprising administering to the subject a therapeutically effect amount of a compound of Formula III:

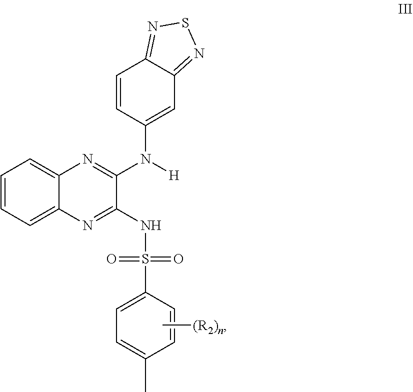

a salt thereof, or a solvate of any of the foregoing wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n is 1, 2, 3, or 4.

In some embodiments, n is 1, 2, or 3. In some embodiments, n is 1.

In some embodiments, R₂ at each occurrence independently is chosen from halogen, hydroxyl, alkoxy, amino, nitro, —CF₃, or —CN. In some embodiments, R₂ at each occurrence independently is chosen from halogen. In some embodiments, R₂ at each occurrence independently is F.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula IV:

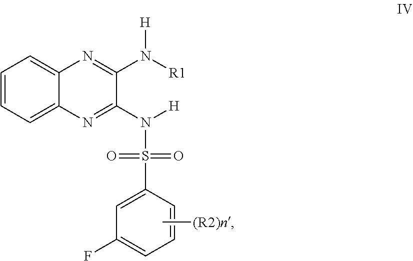

a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; n′ is 0, 1, 2, 3, or 4.

In some embodiments, n′ is 0, 1, or 2. In some embodiments, n′ is 0. In some embodiments, n′ is 1. In some embodiments, n′ is 2.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula V:

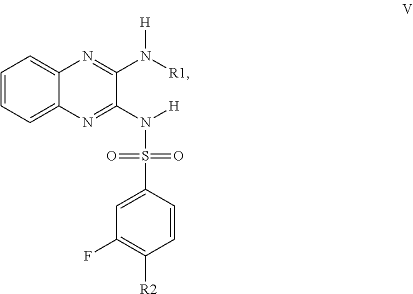

a salt thereof, or a solvate of any of the foregoing;
wherein
R₁ is an aryl group; and
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, the subject suffers from a disease. In some embodiments, the subject suffers from a cancer.

In some embodiments, R₁ is a 5-, 6-, 7-, 8-, 9-, or 10-membered single-ring aromatic group, wherein each of the 5-, 6-, 7-, 8-, 9-, and 10-membered single-ring aromatic groups includes 0, 1, 2, 3, or 4 heteroatom and is optionally substituted with 1, 2, 3, or 4 substituents.

In some embodiments, R₁ is a 6- or 10-membered single-ring aromatic group, wherein each of the 6- and 10-membered single-ring aromatic groups is optionally substituted with 1, 2, 3, or 4 substituents. In some embodiments, R₁ is phenyl optionally substituted with 1, 2, 3, or 4 substituents.

In some embodiments, the present teachings relate to a method of treating a subject in need thereof comprising administering to the subject a therapeutically effect amount of a compound of Formula VI:

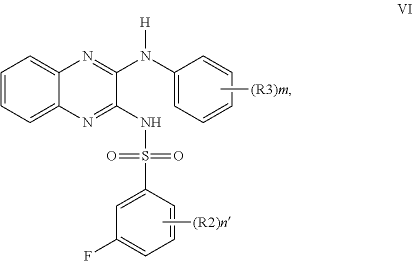

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic;
m is 0, 1, 2, 3, or 4; and
n′ is 0, 1, 2, 3, or 4.

In some embodiments, n′ is 0, 1, or 2. In some embodiments, n′ is 0. In some embodiments, n′ is 1. In some embodiments, n′ is 2.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula VII:

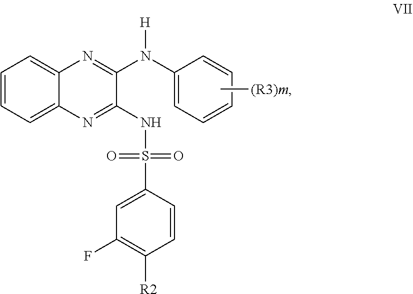

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN, or two R₃ together with the carbon atoms that the two R₃ are attached form a carobocycle or heterocyclic; and
m is 0, 1, 2, 3, or 4.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, m is 2 and R₃ at each occurrence independently is chosen from halogen, alkyl, hydroxyl, alkoxy, amino, nitro, carbonyl, alkylthio, sulfonyl, —CF₃, or —CN. In some embodiments, m is 2 and R₃ at each occurrence independently is chosen from halogen or alkoxy.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula VIII:

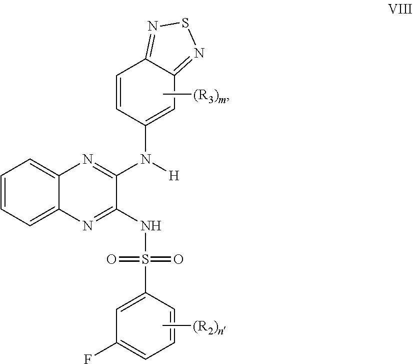

a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
m is 0, 1, 2, 3, or 4; and
n′ is 0, 1, 2, 3, or 4.

In some embodiments, m is 0, 1, 2, or 3. In some embodiments, m is 0. In some embodiments, m is 1. In some embodiments, m is 2.

In some embodiments, R₃ at each occurrence independently is chosen from halogen, azide, alkyl, aralkyl, alkenyl, alkynyl, cycloalkyl, hydroxyl, alkoxy, amino, nitro, carbonyl, carboxyl, silyl, ether, alkylthio, sulfonyl, sulfonamido, ketone, aldehyde, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, nitro, sulfonyl, sulfonamido, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, ester, heterocyclyl, aromatic or heteroaromatic moieties, —CF₃, or —CN.

In some embodiments, n′ is 0, 1, or 2. In some embodiments, n′ is 0. In some embodiments, n′ is 1. In some embodiments, n′ is 2.

In some embodiments, the subject suffers from a disease. In some embodiments, the subject suffers from a cancer.

In some embodiments, the present teachings relate to a method of treating a subject in need thereof comprising administering to the subject a therapeutically effect amount of a compound of Formula IX:

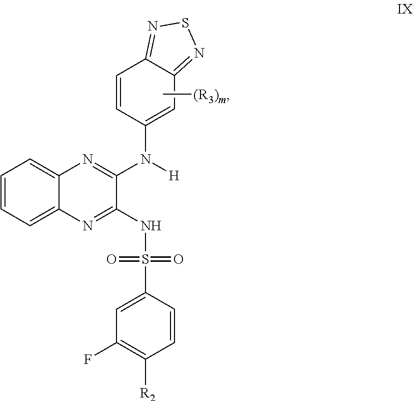

a salt thereof, or a solvate of any of the foregoing;
wherein
R₂ is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
m is 0, 1, 2, 3, or 4.

In some embodiments, R₂ is chosen from halogen, alkyl, aralkyl, alkenyl, alkynyl, cycloalkyl, hydroxyl, alkoxy, amino (or quaternized amino), nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, cycloalkyl, hydroxyl, alkoxy, amino, nitro, amido, carbonyl, carboxyl, silyl, ether, alkylthio, sulfonyl, sulfonamido, ketone, aldehyde, ester, —CF₃, or —CN. In some embodiments, R₂ is chosen from halogen, alkyl, alkoxy, amino, nitro, amido, carbonyl, carboxyl, sulfonyl, sulfonamido, ketone, aldehyde, ester, —CF₃, or —CN. In some embodiments, R₂ is chosen from halogen, alkyl, alkoxy, amino, carboxyl, —CF₃, or —CN. In some embodiments, R₂ is chosen from F, Cl, Br, or I. In some embodiments, R₂ is F. In some embodiments, R₂ is Cl. In some embodiments, R₂ is Br.

In some embodiments, R₂ is optionally substituted alkyl. In some embodiments, R₂ is alkyl optionally substituted with halogen, hydroxyl, alkoxy, amino, nitro, —CF₃, or —CN. In some embodiments, R₂ is alkyl optionally substituted with halogen, hydroxyl, alkoxy, amino, —CF₃, or —CN. In some embodiments, R₂ is chosen from methyl, ethyl, propyl, isopropyl, butyl, or t-butyl, each optionally substituted with one or more substituents, each independently chosen from halogen, hydroxyl, alkoxy, amino, —CF₃, or —CN. In some embodiments, R₂ is chosen from methyl, ethyl, propyl, or isopropyl, each optionally substituted with one or more substituents, each independently chosen from F, Cl, Br, hydroxyl, alkoxy, amino, —CF₃, or —CN. In some embodiments, R₂ is methyl optionally substituted with one or more substituents, each independently chosen from F, Cl, Br, hydroxyl, alkoxy, amino, —CF₃, or —CN. In some embodiments, R₂ is ethyl optionally substituted with one or more substituents, each independently chosen from F, Cl, Br, hydroxyl, alkoxy, amino, —CF₃, or —CN. In some embodiments, R₂ is methyl optionally substituted F, Cl, Br, hydroxyl, alkoxy, amino, —CF₃, or —CN. In some embodiments, R₂ is ethyl optionally substituted with F, Cl, Br, hydroxyl, alkoxy, amino, —CF₃, or —CN. In some embodiments, R₂ is hydroxylmethyl. In some embodiments, R₂ is methyl.

In some embodiments, R₂ is amino optionally substituted with 1, or 2 substituents, each independently chosen from alkyl, alkenyl, alkynyl, aryl, or heteroaryl wherein each of alkyl, alkenyl, alkynyl, aryl, and heteroaryl optionally is further substituted. In some embodiments, R₂ is amino optionally substituted with 1 or 2 substituents, each independently chosen from alkyl, aryl, or heteroaryl, wherein each of alkyl, aryl, and heteroaryl optionally is further substituted. In some embodiments, R₂ is amino. In some embodiments, R₂ is amino substituted with 1 substituent chosen from alkyl, aryl, or heteroaryl, wherein each of alkyl, aryl, and heteroaryl optionally is further substituted. In some embodiments, R₂ is monoalkylamino. In some embodiments, R₂ is chosen from methylamino, ethylamino, propylamino, iso-propylamino, butylamino, iso-butylamino, or tert-butylamino. In some embodiments, R₂ is methylamino (CH₃NH—). In some embodiments, R₂ is ethylamino (CH₃CH₂NH—). In some embodiments, R₂ is isopropylamino ((CH₃)₂CHNH—). In some embodiments, R₂ is amino substituted with 2 substituents each independently chosen from alkyl, aryl, or heteroaryl, wherein each of alkyl, aryl, and heteroaryl optionally is further substituted. In some embodiments, R₂ is amino substituted with 2 substituents each independently chosen from methyl, ethyl, propyl, isopropyl, butyl, iso-butyl, or tert-butyl, wherein each of methyl, ethyl, propyl, isopropyl, butyl, iso-butyl, and tert-butyl optionally is further substituted.

In some embodiments, R₂ is alkoxy. In some embodiments, R₂ is Rₐ—O—, wherein Rₐ is chosen alkyl, alkenyl, alkynyl, aryl, or heteroaryl, wherein each of alkyl, alkenyl, alkynyl, aryl, and heteroaryl optionally is further substituted. In some embodiments, R₂ is chosen from methoxy, ethoxy, isopropoxy, or tert-butoxy. In some embodiments, R₂ is methoxy. In some embodiments, R₂ is ethoxy. In some embodiments, R₂ is isopropoxy.

In some embodiments, R₂ is chosen from nitro, amido, carbonyl, sulfonyl, sulfonamido, —CF₃, or —CN. In some embodiments, R₂ is nitro. In some embodiments, R₂ is amido as defined herein. In some embodiments, R₂ is carbonyl as defined herein. In some embodiments, R₂ is carboxylic acid. In some embodiments, R₂ is ester as defined herein. In some embodiments, R₂ is sulfonyl. In some embodiments, R₂ is sulfonamido. In some embodiments, R₂ is aldehyde. In some embodiments, R₂ is ester. In some embodiments, R₂ is —CF₃ or —CN.

In some embodiments, m is 0, 1, 2, or 3. In some embodiments, m is 0. In some embodiments, m is 1. In some embodiments, m is 2.

In some embodiments, R₃ at each occurrence independently is chosen from halogen, azide, alkyl, aralkyl, alkenyl, alkynyl, cycloalkyl, hydroxyl, alkoxy, amino, nitro, carbonyl, carboxyl, silyl, ether, alkylthio, sulfonyl, sulfonamido, ketone, aldehyde, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, nitro, sulfonyl, sulfonamido, ester, heterocyclyl, aromatic or heteroaromatic moieties, —CF₃, or —CN. In some embodiments, R₃ at each occurrence independently is chosen from halogen, alkyl, cycloalkyl, hydroxyl, alkoxy, amino, ester, heterocyclyl, aromatic or heteroaromatic moieties, —CF₃, or —CN.

In some embodiments, the subject suffers from a disease. In some embodiments, the subject suffers from a cancer. In some embodiments, the cancer is chosen from colon adenocarcinoma, rectal adenocarcinoma, gastric adenocarcinoma, gastroesophageal junction adenocarcinoma, esophageal adenocarcinoma, hepatocellular carcinoma, ovarian cancer, platinum-resistant ovarian cancer, pancreatic adenocarcinoma, breast cancer, triple negative breast cancer, ovarian cancer, cholangiocarcinoma, melanoma, small cell lung cancer, and non-small cell lung cancer. In some embodiments, the cancer is colorectal cancer. In some embodiments, the cancer is a lung cancer. In some embodiments, the cancer is small cell lung cancer. In some embodiments, the cancer is non-small cell lung cancer. In some embodiments, the cancer is breast cancer.

In some embodiments, the present teachings relate to a method of treating a subject in need thereof comprising administering to the subject a therapeutically effect amount of a compound chosen from:

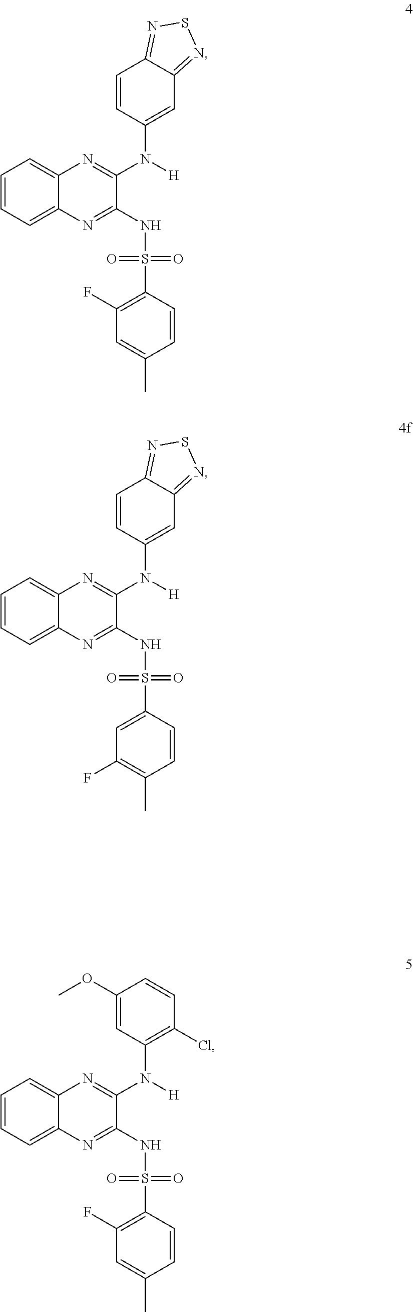 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F ; COc1ccc(Cl)c(Nc2nc3ccccc3nc2NS(=O)(=O)c2ccc(C)cc2F)c1]

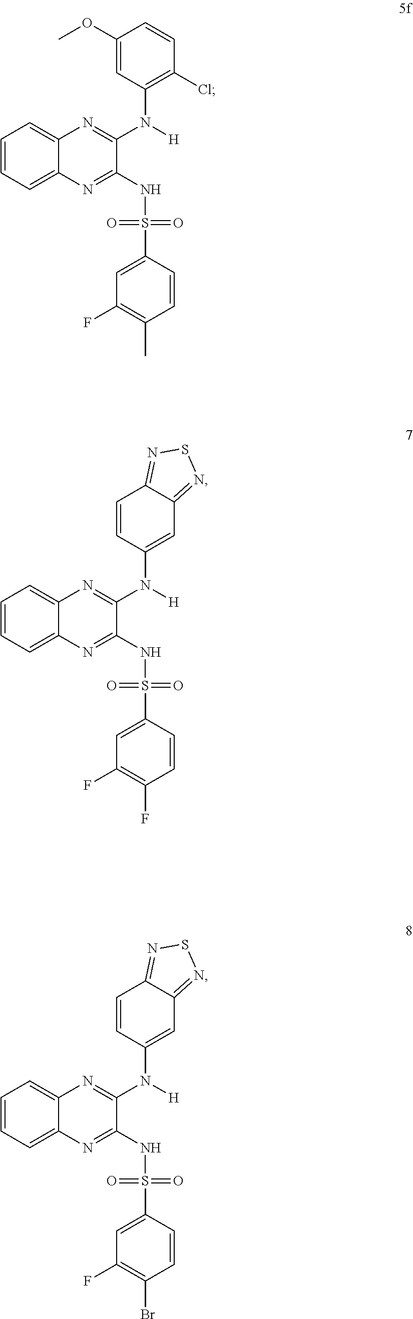 [SMILES: COc1ccc(Cl)c(Nc2nc3ccccc3nc2NS(=O)(=O)c2ccc(C)c(F)c2)c1 ; O=S(=O)(Nc1nc2ccccc2nc1Nc1ccc2nsnc2c1)c1ccc(F)c(F)c1 ; O=S(=O)(Nc1nc2ccccc2nc1Nc1ccc2nsnc2c1)c1ccc(Br)c(F)c1]

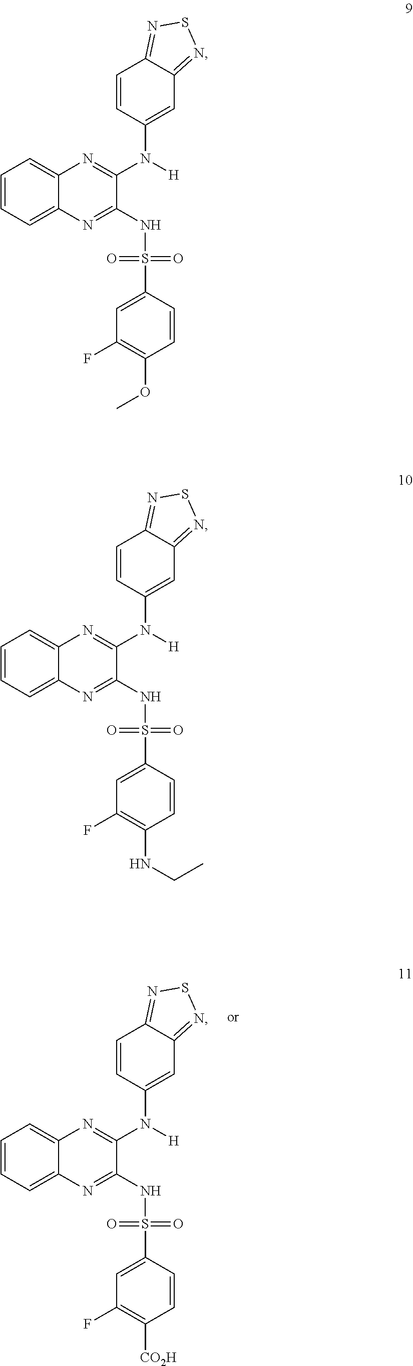 [SMILES: COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F ; O=C(O)c1ccc(S(=O)(=O)Nc2nc3ccccc3nc2Nc2ccc3nsnc3c2)cc1F ; "or"]

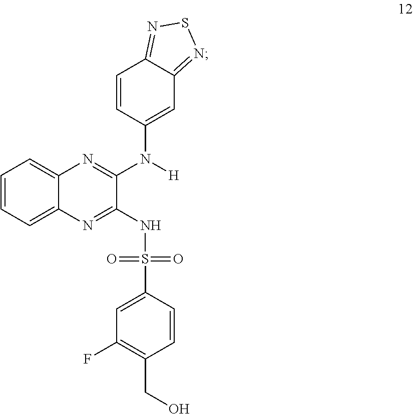 [SMILES: O=S(=O)(Nc1nc2ccccc2nc1Nc1ccc2nsnc2c1)c1ccc(CO)c(F)c1]

a salt thereof, or a solvate of any of the foregoing.

the present teachings relate to a method of treating a subject in need thereof comprising administering to the subject a therapeutically effect amount of a compound chosen from:

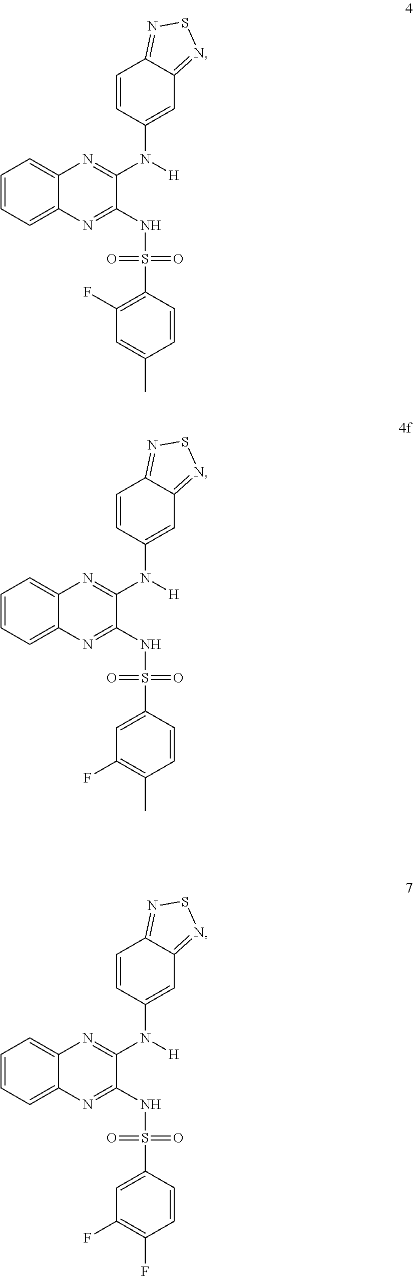 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F ; O=S(=O)(Nc1nc2ccccc2nc1Nc1ccc2nsnc2c1)c1ccc(F)c(F)c1]

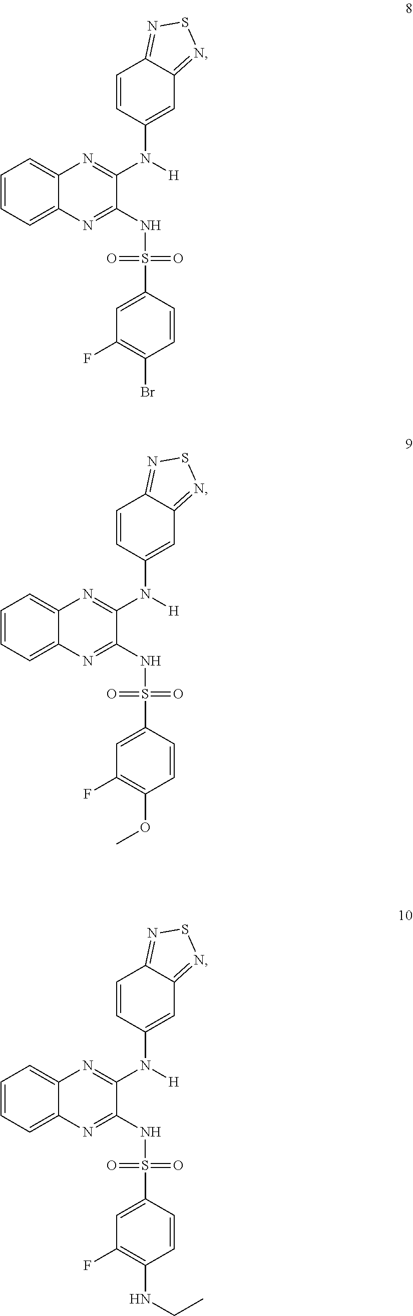 [SMILES: O=S(=O)(Nc1nc2ccccc2nc1Nc1ccc2nsnc2c1)c1ccc(Br)c(F)c1 ; COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F]

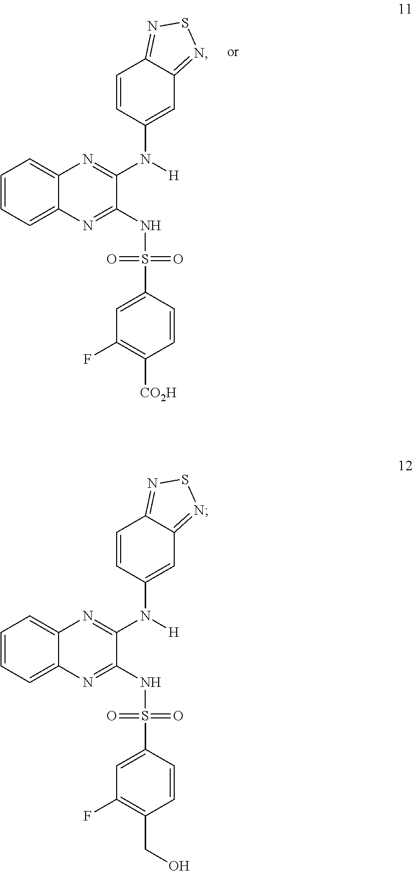 [SMILES: O=C(O)c1ccc(S(=O)(=O)Nc2nc3ccccc3nc2Nc2ccc3nsnc3c2)cc1F ; "or" ; O=S(=O)(Nc1nc2ccccc2nc1Nc1ccc2nsnc2c1)c1ccc(CO)c(F)c1]

a salt thereof, or a solvate of any of the foregoing.

In some embodiments, the present teachings relate to a method of treating a subject in need thereof comprising administering to the subject a therapeutically effect amount of a compound of Formula 4f:

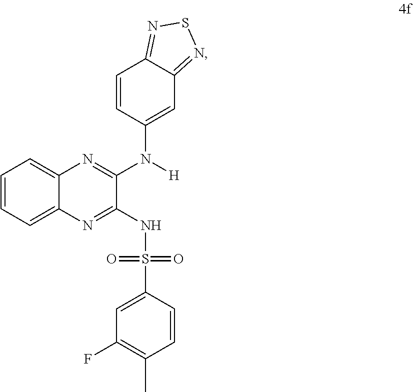 [SMILES: Cc1ccc(S(=O)(=O)Nc2nc3ccccc3nc2Nc2ccc3nsnc3c2)cc1F]

a salt thereof, or a solvate of any of the foregoing.

In some embodiments, the present teachings relate to a method of treating a subject in need thereof comprising administering to the subject a therapeutically effect amount of a compound of Formula 7:

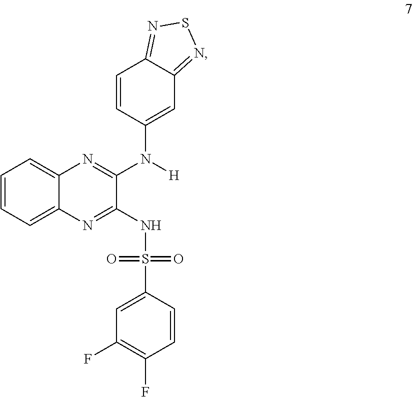 [SMILES: O=S(=O)(Nc1nc2ccccc2nc1Nc1ccc2nsnc2c1)c1ccc(F)c(F)c1]

a salt thereof, or a solvate of any of the foregoing.

In some embodiments, the subject suffers from a disease. In some embodiments, the subject suffers from a cancer. In some embodiments, the cancer is chosen from colon adenocarcinoma, rectal adenocarcinoma, gastric adenocarcinoma, gastroesophageal junction adenocarcinoma, esophageal adenocarcinoma, hepatocellular carcinoma, ovarian cancer, platinum-resistant ovarian cancer, pancreatic adenocarcinoma, breast cancer, triple negative breast cancer, ovarian cancer, cholangiocarcinoma, melanoma, small cell lung cancer, and non-small cell lung cancer. In some embodiments, the cancer is colorectal cancer. In some embodiments, the cancer is a lung cancer. In some embodiments, the cancer is small cell lung cancer. In some embodiments, the cancer is non-small cell lung cancer. In some embodiments, the cancer is breast cancer.

In one aspect, the present teachings relate to a method of preparing a compound of the present teachings.

The following examples are intended to illustrate certain embodiments of the present teachings, do not exemplify the full scope of the present teachings, and therefore should not be construed to limit the scope of the present teachings.

### Example 1: Preparation of 2-fluoro-4-methylbenzenesulfonamide 2

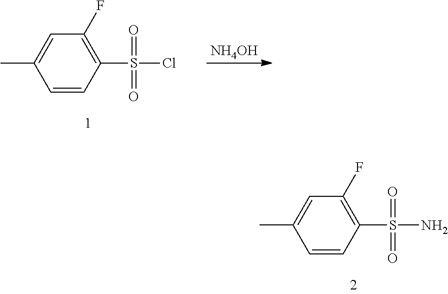

2-Fluoro-4-methylbenzene-1-sulfonyl chloride 1 (0.2 g, 0.96 mmol) was suspended in concentrated ammonia (5 mL) and the mixture was agitated at room temperature for 4 hours. The reaction mixture was extracted with ethyl acetate twice (5× and 10 mL). The organic layers were combined, washed with water, and saturated brine, dried, and concentrated to provide 2-fluoro-4-methlbenzene-1-sufonamide 2 (205 mg, white solid, 100% yield).

### Example 2: Preparation of N-(3-chloro-quinoxalin-2-yl)-2-fluoro-4-methyl-benzenesufonamide 3

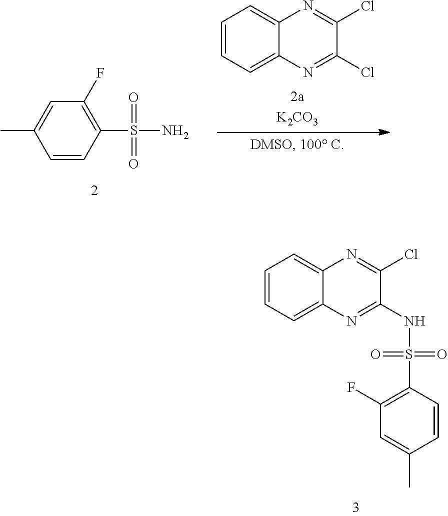

2-Fluoro-4-methlbenzene-1-sufonamide 2 (0.380 g, 2.0 mmol) and 2,3-dichloro-quinoxaline 2a (0.402 g, 2.02 mmol) were suspended in DMSO (11.4 mL) and the mixture was agitated at 100° C. for 240 minutes. After the reaction was found complete according to TLC (petroleum ether:ethyl acetate (1:1)), the reaction mixture was transferred to ice water with 1% acetic acid (114 mL). The solid was filtered, washed, and dried under vacuum to provide N-(3-chloro-quinoxalin-2-yl)-2-fluoro-4-methyl-benzenesufonamide 3 as a white solid (72.3% yield). LC-MS: 352 [M+1]+.

### Example 3: Preparation of N-[3-(benzo[1,2,5]thiadiazol-5-ylamino)-quinoxalin-2-yl]-2-fluoro-4-methyl-benzenesulfonamide 4

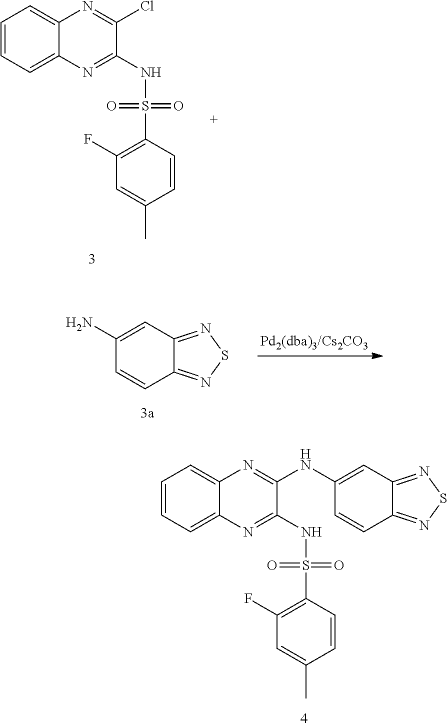 [SMILES: Cc1ccc(S(=O)(=O)Nc2nc3ccccc3nc2Cl)c(F)c1 ; Nc1ccc2nsnc2c1 ; Cc1ccc(S(=O)(=O)Nc2nc3ccccc3nc2Nc2ccc3nsnc3c2)c(F)c1]

N-(3-Chloro-quinoxalin-2-yl)-2-fluoro-4-methyl-benzenesufonamide 3 (0.100 g, 0.28 mmol) and benzo[1,2,5]thiadiazol-5-ylamine 3a (0.045 g, 0.29 mmol), CsCO₃ (0.280 g), and x-phos (0.025 g) were suspended in 1,4-dioxane. Under N₂ blanket, Pd₂(dba)₃ (0.025 g) was added and the mixture was stirred at 110° C. for 240 minutes. After the reaction was found complete according to TLC. Water (20 mL) and ethyl acetate (20 mL) were added and the mixture was stirred. After partition, the aqueous layer was extracted with ethyl acetate twice. All the ethyl acetate solutions were combined, washed with saturated brine, and dried. The filtered ethyl acetate was concentrated and the residue was separated with silica gel chromatography (ethyl acetate) to provide N-[3-(benzo[1,2,5]thiadiazol-5-ylamino)-quinoxalin-2-yl]-2-fluoro-4-methyl-benzenesulfonamide 4 as a yellow solid (50 mg, 38.4% yield). LC-MS: 467[M+1]+. ¹H NMR (400 MHz, DMSO-d6) δ 9.32 (d, J=1.7 Hz, 1H), 9.23 (s, 1H), 8.11-7.84 (m, 3H), 7.64-7.51 (m, 1H), 7.33-7.11 (m, 3H), 7.07 (d, J=7.9 Hz, 1H), 6.94 (d, J=11.3 Hz, 1H), 2.30 (s, 3H).

### Example 4: Preparation of 3-fluoro-4-methyl-benzenesulfonamide 2f

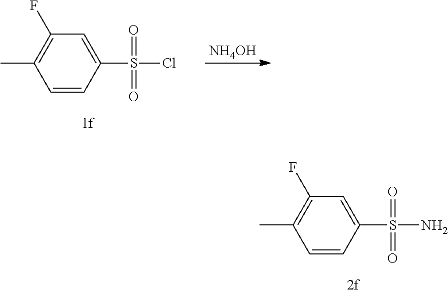

3-Fluoro-4-methylbenzene-1-sulfonyl chloride if (0.5 g, 2.4 mmol) was suspended in concentrated ammonia (10 mL) and the mixture was agitated at room temperature for 4 hours. After the reaction was complete according to TLC (PE/EA=3/1), the reaction mixture was extracted with ethyl acetate twice (5× and 10 mL). The organic layers were combined, washed with water, and saturated brine, dried, and concentrated to provide 3-fluoro-4-methlbenzene-1-sufonamide 2f as a white solid (380 mg, 100% yield). LC-MS: 190 [M+1]+.

### Example 5: Preparation of N-(3-chloro-quinoxalin-2-yl)-3-fluoro-4-methyl-benzenesufonamide 3f

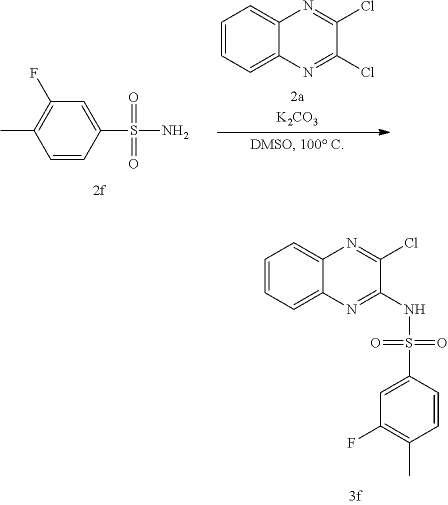

3-Fluoro-4-methlbenzene-1-sufonamide 2f (0.380 g, 1.0 mmol) and 2,3-dichloro-quinoxaline 2a (0.402 g, 2.02 mmol) were suspended in DMSO (11.4 mL) and the mixture was agitated at 100° C. for 240 minutes. After the reaction was found complete according to TLC (petroleum ether:ethyl acetate (1:1)), the reaction mixture was transferred to ice water with 1% acetic acid (114 mL). The solid was filtered, washed, and dried under vacuum to provide N-(3-chloro-quinoxalin-2-yl)-3-fluoro-4-methyl-benzenesufonamide 3f as a white solid (0.509 g, 72.3% yield). LC-MS: 352 [M+1]+.

### Example 6: Preparation of N-[3-(benzo[1,2,5]thiadiazol-5-ylamino)-quinoxalin-2-yl]-3-fluoro-4-methyl-benzenesulfonamide 4f

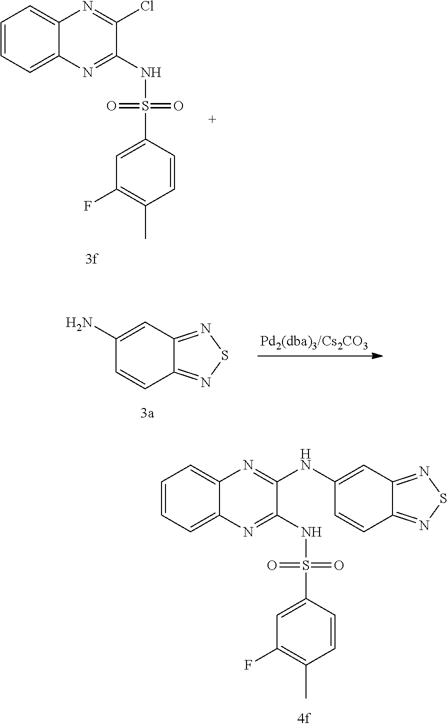 [SMILES: Cc1ccc(S(=O)(=O)Nc2nc3ccccc3nc2Cl)cc1F ; Nc1ccc2nsnc2c1 ; Cc1ccc(S(=O)(=O)Nc2nc3ccccc3nc2Nc2ccc3nsnc3c2)cc1F]

N-(3-Chloro-quinoxalin-2-yl)-3-fluoro-4-methyl-benzenesufonamide 3f (0.509 g, 1.45 mmol) and benzo[1,2,5]thiadiazol-5-ylamine 3a (0.230 g, 1.52 mmol), CsCO₃ (1.42 g), and x-phos (0.207 g) were suspended in 1,4-dioxane (25 mL). Under N₂ blanket, Pd₂(dba)₃ (0.133 g) was added and the mixture was stirred at 110° C. for 240 minutes. After the reaction was found complete according to TLC. Water (40 mL) and ethyl acetate (40 mL) were added and the mixture was stirred. After partition, the aqueous layer was extracted with ethyl acetate twice. All the ethyl acetate solutions were combined, washed with saturated brine, and dried. The filtered ethyl acetate was concentrated and the residue was separated with silica gel chromatography (ethyl acetate) to provide N-[3-(benzo[1,2,5]thiadiazol-5-ylamino)-quinoxalin-2-yl]-3-fluoro-4-methyl-benzenesulfonamide 4f as a greenish yellow solid (506 mg, 74.8% yield). LC-MS: 467[M+1]+. ¹H NMR (400 MHz, DMSO-d6) δ 12.40 (s, 1H), 9.45 (s, 1H), 9.14 (s, 1H), 8.32-7.91 (m, 4H), 7.91-7.83 (m, 1H), 7.77-7.67 (m, 1H), 7.54 (t, J=7.7 Hz, 1H), 7.49-7.38 (m, 2H), 2.31 (s, 3H).

By starting with the corresponding starting material and using similar procedures described in Examples 1-6, Compounds 6-9 were prepared as follows:

| | LC-MS/ | |
|---|---|---|
| Compound | M + 1 | ¹H NMR (600 MHz, DMSO) |
| 6 | 453 | δ12.44 (s, 1H), 9.45 (s, 1H), 9.16 (s, 1H), |
| | | 8.09 (s, 1H), 8.06-8.00 (m, 2H), 7.97 (d, J = |
| | | 7.8 Hz, 1H), 7.68 (d, J = 36.4 Hz, 2H), |
| | | 7.47 (d, J = 40.3 Hz, 3H) |
| 7 | 471 | δ 12.46 (s, 1H), 9.45 (s, 1H), 9.14 (s, 1H), |
| | | 8.31 (dd, J = 12.8, 4.8 Hz, 1H), 8.11 (d, J = |
| | | 9.3 Hz, 1H), 8.03-7.96 (m, 2H), 7.96 (s, 1H), |
| | | 7.76-7.71 (m, 1H), 7.71-7.67 (m, 1H), |
| | | 7.48-7.42 (m, 2H) |
| 8 | 533 | δ 9.46 (d, J = 21.8 Hz, 1H), 9.14 (d, J = |
| | | 14.3 Hz, 1H), 8.21-8.07 (m, 2H), 8.04 (m, |
| | | 1H), 8.01-7.87 (m, 3H), 7.73 (d, J = 4.9 |
| | | Hz, 1H), 7.70-7.62 (m, 1H), 7.51-7.39 (m, |
| | | 2H) |
| 9 | 483 | δ 12.32 (s, 1H), 9.44 (s, 1H), 9.13 (s, 1H), |
| | | 8.10 (s, 1H), 8.05 (dd, J = 17.5, 5.7 Hz, |
| | | 2H), 7.96 (d, J = 8.5 Hz, 2H), 7.73-7.68 |
| | | (m, 1H), 7.49-7.40 (m, 2H), 7.37 (t, J = |
| | | 8.5 Hz, 1H), 3.92 (s, 3H) |

### Example 7: Cell Culture and Cytotoxicity Assay

The assayed compounds each was dissolved in DMSO with a final concentration of 10 mM or 40 mM and stored at −20° C. Tumor cell lines A549 (7,000 cells/well), BT474 (12,000 cells/well), MDA-MB-468 (12,000 cells/well), and HCC1937 (6,000 cells/well) were seeded on 96-well plates. And the cells were pre-incubated at 37° C./5% CO₂ for 24 hours. The cells were exposed to different concentrations of the assay compounds (from 400 μM to 782 nM, from 200 μM to 391 nM, from 100 μM to 196 nM, and from 50 μM to 98 nM) for the following time:

| Cell lines | Exposure period (/hour) |
|---|---|
| A549 | 72 |
| BT474 | 96 |
| MDA-MB-468 | 72 |
| HCC1937 | 72 |

After incubation, 100 μL from each well was discarded and CTG base (Proega, G7571, 50 μL) was added and the mixture was agitated for 2 minutes. The mixtures were stored in a dark room for 10 minutes and the viability was analyzed with Multiskan microplate reader.

The IC50 of the assayed compounds are as shown in Tables 1 and 2. As a comparison, the following compounds were used as references.

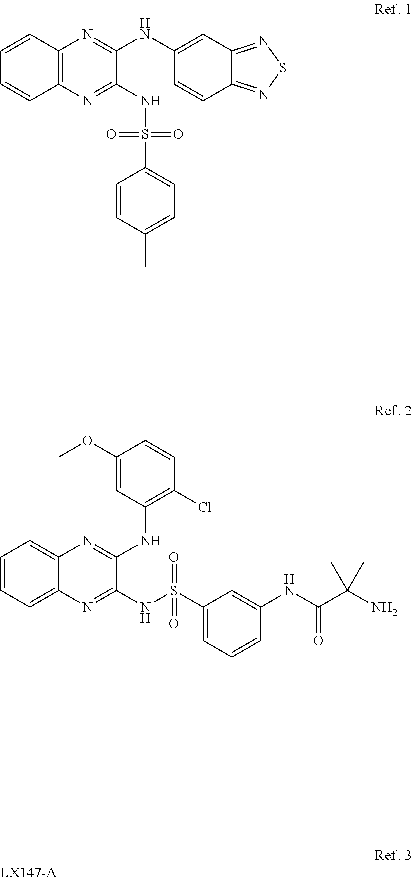 [SMILES: Cc1ccc(S(=O)(=O)Nc2nc3ccccc3nc2Nc2ccc3nsnc3c2)cc1 ; COc1ccc(Cl)c(Nc2nc3ccccc3nc2NS(=O)(=O)c2cccc(NC(=O)C(C)(C)N)c2)c1]

TABLE 1

| | IC50 (nM) | |
|---|---|---|
| Compound | A549 | BT474 |
| Ref. 1 | >50000 | 28187 |
| 4 | NA | >50000 |
| 4f | 50000 > IC50 > 25000 | 25000 > IC50 > 12500 |
| Ref. 2 | 50000 > IC50 > 25000 | 50000 > IC50 > 25000 |
| Ref. 3 | >50000 | >50000 |

TABLE 2

| | IC50 (nM) | IC50 (nM) |
|---|---|---|
| Compound | MDA-MB-468 | HCC1937 |
| Ref. 1 | 2059 | NA |
| 4 | 4252 | 114199 |
| 4f | 1562.5 > IC50 > 781.25 | 37911 |
| Ref. 2 | 25000 > IC50 > 12500 | 100000 > IC50 > 50000 |
| Ref. 3 | >50000 | NA |

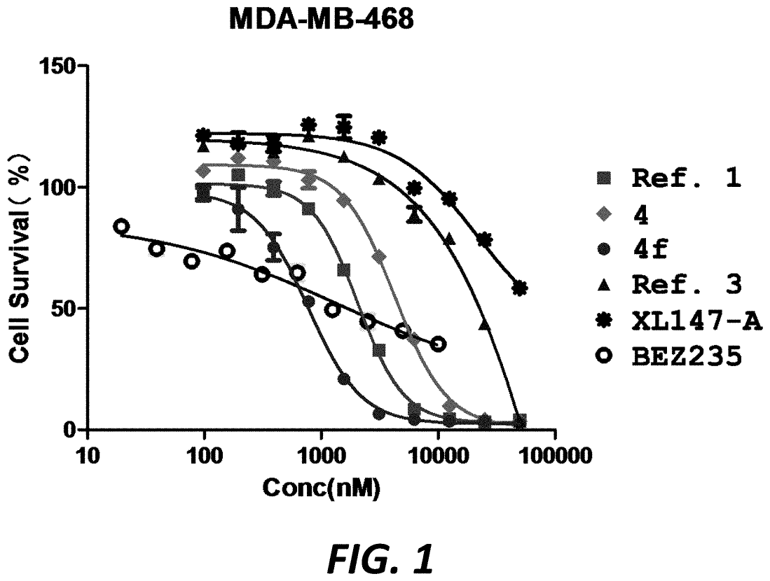

In addition, as shown in FIG. 1, Compound 4f has a much better anticancer activity than any of the known compounds (e.g., Ref. 1, Ref. 3, XL147-A, and BEZ235).

### Example 8: Cytotoxicity Assay by Using the CTG Method

Cell Seeding:

The corresponding cells were harvested from flask into cell culture medium and the cell number were counted. The cells were diluted with culture medium to the desired density and 40 μL of cell suspension was added into each well of 384-well cell culture plate as designated. The plates were covered with lid and placed in room temperature for 30 minutes without shaking and then were transferred plates into 37° C. 5% CO2 incubator overnight for cell attachment.

Compound preparation and treatment: Test compounds were received or dissolved at 30 mM stock solution and control compound Staurosporine was stored as 10 mM and 1 mM concentration. 45 uL of stock solution was transferred to a 384 pp-plate. 3 fold, 10-point dilution were performed via transferring 15 uL compound into 30 μL DMSO by using TECAN (EVO200) liquid handler. The plates were spun at room temperature at 1,000 RPM for 1 minute and shaken at a plate shaker for 2 minutes.

40 nL of diluted compound were transferred from the compound source plate into the cell plate as designated by using Echo550 and then the plates were placed back to incubator.

On Day 3 (72 h after adding the compound), CTG detection for compound treatment plates were performed as described in “Detection” section.

Detection:

The plate from incubators were placed and equilibrated at room temperature for 15 minutes. The CellTiter Glo reagents were thawed and equilibrated to room temperature before the experiment. 40 μL of CellTiter-Glo reagent were transferred into each well to be detected (at 1:1 to culture medium). Then the plates were placed at room temperature for 30 min followed by reading on EnSpire.

Data Analysis:

The inhibition activity was calculated following the formula below:

Inhibition (%)=100%×(Lum_{vehicle}−Lumₛₐₘₚₗₑ)/(Lum_{vehicle}−Lum_{blank})

The IC50 were calculated by fitting the Curve using Xlfit (v5.3.1.3):

Y=Bottom+(Top−Bottom)/(1+10\hat{ }((Log IC50−X)*HillSlope)).

TABLE 3
in vitro assay results of 4f:

| Cell Line | Cancer Type | IC50 (nM) |
|---|---|---|
| MCF-7 | Breast cancer | 5194.77 |
| MDA-MB-468 | Breast cancer | 2488.71 |
| T-47D | Breast cancer | 5743.01 |
| BT-474 | Breast cancer | 7013.17 |
| MDA-MB-453 | Breast cancer | 1503.69 |
| ZR-75-1 | Breast cancer | 1530.98 |
| COLO205 | Colon cancer | 2993.29 |
| HCT116 | Colon cancer | 5436.96 |
| HT29 | Colon cancer | 2441.38 |
| LoVo | Colon cancer | 21703.07 |
| A498 | Kidney cancer | 3679.77 |
| CaKi-1 | Kidney cancer | 12985.88 |
| 786-O | Kidney cancer | 5743.01 |
| MOLT4 | Leukemia | 4717.27 |
| HL-60 | Leukemia | 7610.34 |
| MV-4-11 | Leukemia | 6796.34 |
| Hep3B | Liver cancer | 3973.83 |
| PLC/PRF/5 | Liver cancer | 3265.85 |
| A549 | Lung cancer | 8326.58 |
| Calu-6 | Lung cancer | 4894.46 |
| NCI-H446 | Lung cancer | 3924.39 |
| U937 | Lymphoma | 3679.77 |
| WM-266-4 | Melanoma | 9099.11 |
| A2058 | Melanoma | 7696.71 |
| SK-MEL-3 | Melanoma | 9060.40 |
| SK-OV-3 | Ovarian cancer | 9374.99 |
| OVCAR-3 | Ovarian cancer | 6681.57 |
| PC-3 | Prostate cancer | 6050.32 |
| LNCaP | Prostate cancer | 3200.09 |
| U87MG | Glioblastoma | 17981.41 |

### Example 9: Anti-Tumor Activity Against MDA-MB-468 Cell Line

Cell Culture Medium

TABLE 4
Cell culture conditions and inoculation density

| | | Inoculation density |
|---|---|---|
| Cell Line | Medium | (cell/well) |
| MDA-MB-468 | L15 + 10% FBS | 5000 |

Cell Thawing.

A cryo tube was removed from liquid nitrogen. As quickly as possible, the cells were thawed by placing the vial in a 37° C. water bath and gently shaking the vials. The cells were transferred to medium tube, which containing 5 mL medium, and were centrifuged at 1000 rpm for 6 min. The supernatant containing dead cells and cell debris was discarded. 1 mL of pre-warmed medium with 10% FBS was add and the solution was re-suspended. The plate was placed in 37° C. and 5% CO₂ incubator. The medium was changed next day.

Cell Passage.

Cells in logarithmic growth stage were taken and culture medium was discarded. 1 mL 0.25% trypsin solution was added for twice. The trypsin solution was removed and the 0.5 ml 0.25% trypsin solution was added to the cell flask. The plate was placed in 37° C. and 5% CO₂ incubator for a few minutes until the cells recovered. 1 mL medium was added to stop digestion. The liquid was transferred by pipette for experiment.

Cell Plating.

the cells were suspended and the medium was discarded. 1 mL 0.25% trypsin solution was added for twice and the trypsin digestion was centrifuged. The cells were suspended with the culture medium, count and adjusted to the appropriate concentration.

The cell suspension was added to the 96-well plate with a volume of 100 uL per well, as shown in Tab. 5, and was placed in 37° C., 5% CO₂ incubator overnight.

Preparation of Compounds Working Solution.

The test compounds were dissolved in DMSO at 10 mM with sterile filter (0.22 um, nelon, JET). After filtering, the solution were continuously diluted by three times with DMSO. A total of six concentration were prepared, as shown below.

| Row | B | C | D | E | F | G |
|---|---|---|---|---|---|---|
| Conc (uM) | 100 | 33 | 11 | 3 | 1 | 0.3 |

6 uL compound solution was added to 594 uL medium and the solution was added to 96-well plate as shown and cultured for 72 h in CO₂ incubator.

CCK-8 Tests.

A CCK-8 solution was prepared according to the instructions and the 96-well plate was taken out and the original medium discarded. 100 uL cck-8 solution was added to each well in the 96-well plate. Photoabsorption data were read by TECAN enzyme marker after a period of time.

Data Analysis.

Inhibition Equation:

IC (%)=[Ac-As]/[Ac-Ab]*100%
As: Experimental well (containing culture medium, cell, CCK-8 and compound);
Ac: Controlled well (containing culture medium, cell and CCK-8);
Ab: Blank well (Only containing medium and CCK-8).

The Result of anti-tumor activity against MDA-MB-468 cell line was shown in Table 5.

TABLE 5
anti-tumor activity against MDA-MB-468 cell line

| Compound ID | Ref. 1 | 4 f | Ref. 2 | 6 |
|---|---|---|---|---|
| IC 50(uM) | 2.668 | 0.9116 | 4.914 | 2.303 |
| Compound ID | 7 | 8 | 9 | 10 |
| IC 50(uM) | 0.4688 | 0.8106 | 1.395 | 7.093 |

### Example 10: In Vivo Study

MDA-MB-468 cells (human breast cancer) in its log growth phase were implanted in BALB/c-nu mice. When the tumor volume was approximately 50-150 mm³, the mice were randomized into four groups, each include 8 mice. The four groups were treated with vehicle, Ref. 1, 4f, and Ref. 2, respectively, and the tumor sizes were measured two to three times each week and the mice were also weighted.

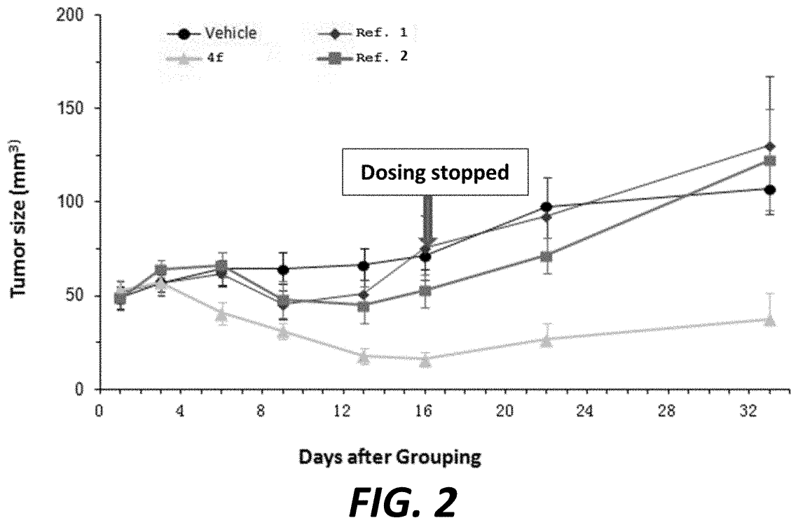

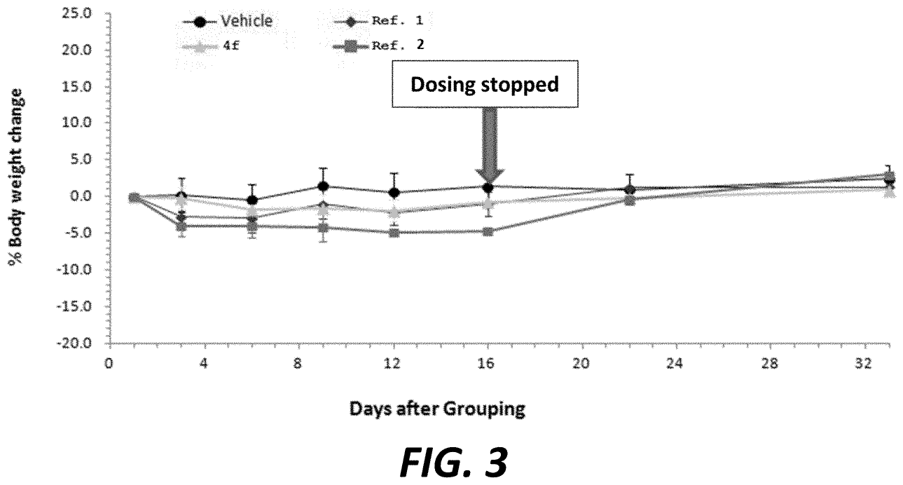

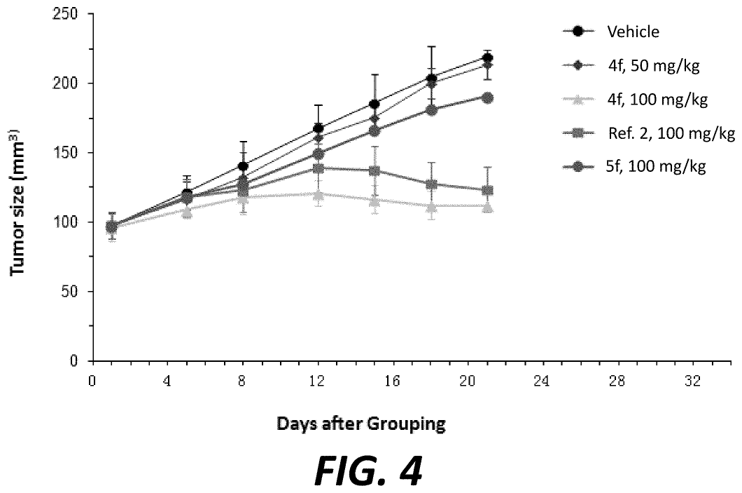

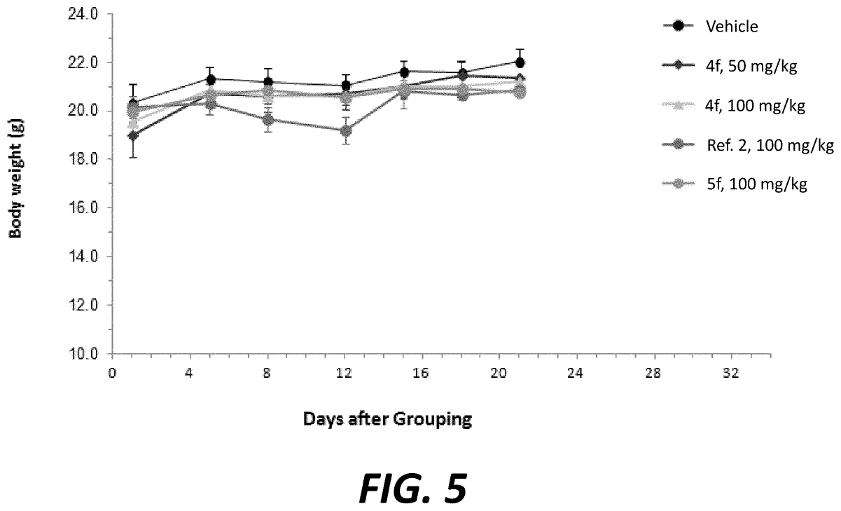

As shown in FIGS. 2 and 4, the results show that there is no significant difference between Ref. 1 and vehicle, and Ref. 2 and vehicle. 4f showed significant anticancer activities. In comparison, as shown in FIGS. 3 and 5, no significant weight changes were observed among the mice dosed with vehicle, Ref. 1, Ref. 2, and 4f.

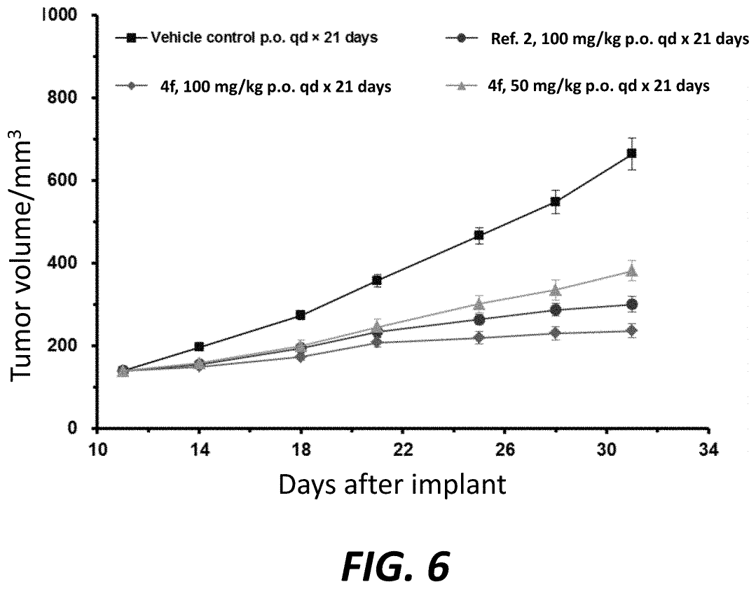

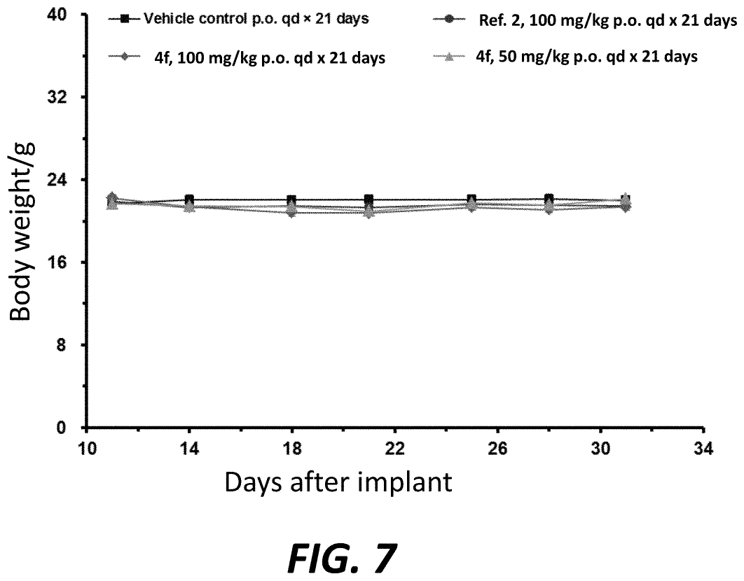

MDA-MB-453 cells (human breast cancer) were also studied in a mouse model. As shown in FIG. 6, at the similar dosage (for example, 100 mg/Kg), compound 4f showed better anticancer activities than Ref. 2 and no difference in weight changes was observed among the groups treated with vehicle, Ref. 2, 4f at 50 mg/Kg, and 4f at 100 mg/Kg, respectively. FIG. 7.

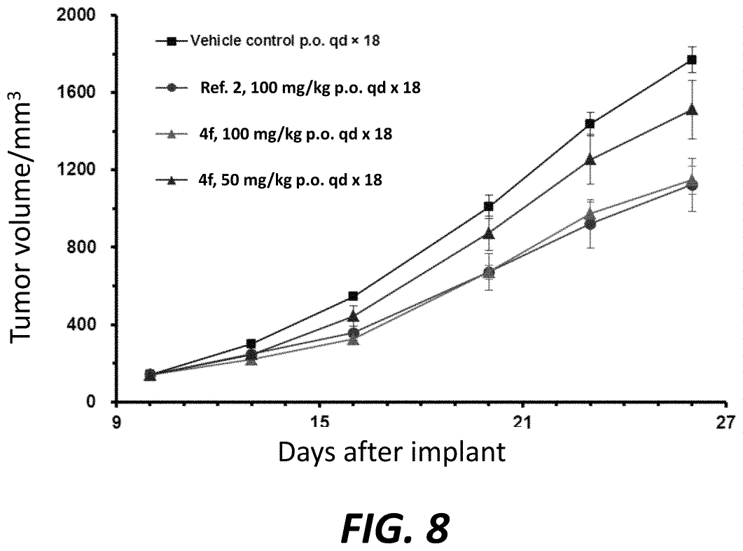

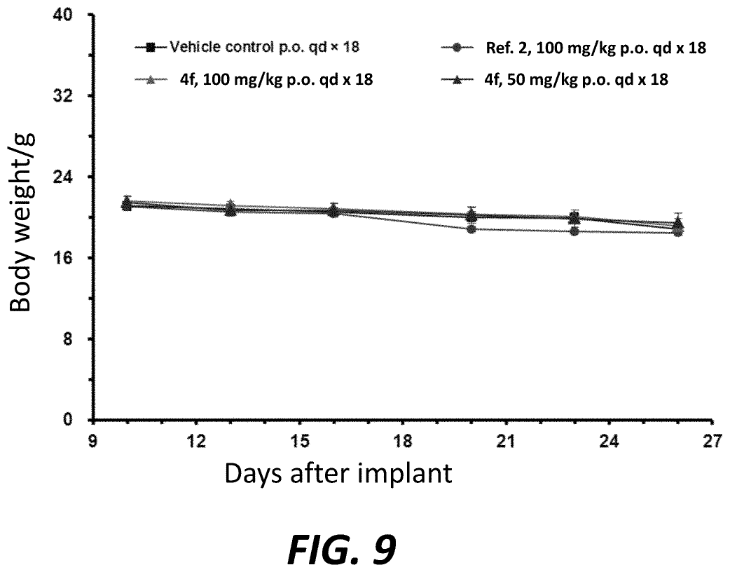

Hep3B cells (human liver cancer) were also studied in a mouse model. As shown in FIG. 8, at the similar dosage (for example, 100 mg/Kg), compound 4f showed similar anticancer activities with Ref. 2 and no difference in weight changes was observed among the groups treated with vehicle, Ref. 2, 4f at 50 mg/Kg, and 4f at 100 mg/Kg, respectively. FIG. 9.

While several embodiments of the present teachings have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present teachings. More generally, those skilled in the art will readily appreciate that all parameters, dimensions, materials, and configurations described herein are meant to be exemplary and that the actual parameters, dimensions, materials, and/or configurations will depend upon the specific application or applications for which the present teachings is/are applied. Those skilled in the art will recognize, or be able to ascertain using no more than routine experimentation, many equivalents to the specific embodiments of the present teachings described herein. It is, therefore, to be understood that the foregoing embodiments are presented by way of example only and that, within the scope of the appended claims and equivalents thereto, the present teachings may be practiced otherwise than as specifically described and claimed. The present teachings are directed to each individual feature and/or method described herein. In addition, any combination of two or more such features and/or methods, if such features and/or methods are not mutually inconsistent, is included within the scope of the present teachings.

The above description of the disclosed embodiments is provided to enable any person skilled in the art to make or use the present teachings disclosed herein. Various modifications to these embodiments will be readily apparent to those skilled in the art, and the generic principles described herein can be applied to other embodiments without departing from the spirit or scope of the present teachings. Thus, it is to be understood that the description and drawings presented herein are representative of the subject matter which is broadly contemplated by the present teachings. It is further understood that the scope of the present teachings is not intended to be limited to the embodiments shown herein but is to be accorded with the widest scope consistent with the patent law and the principles and novel features disclosed herein.

Alternative embodiments of the claimed disclosure are described herein. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the present teachings to be practiced otherwise than as specifically described herein.

Accordingly, this present teachings include all modifications and equivalents of the subject matter recited in the claims appended hereto as permitted by applicable law. Moreover, any combination of the above described elements in all possible variations thereof is encompassed by the present teachings unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. The compound of claim 37, wherein
the compound has Formula VIII: 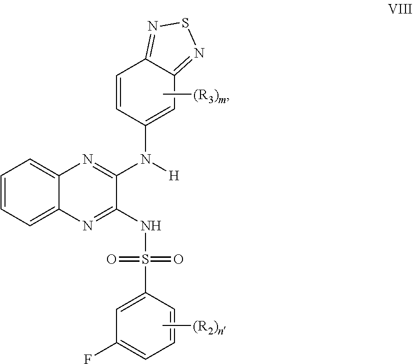
a salt thereof, or a solvate of any of the foregoing;
wherein
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN;
m is 0, 1, 2, 3, or 4; and
n′ is 0, 1, 2, 3, or 4.

2. The compound of claim 1, wherein the compound has Formula IX: 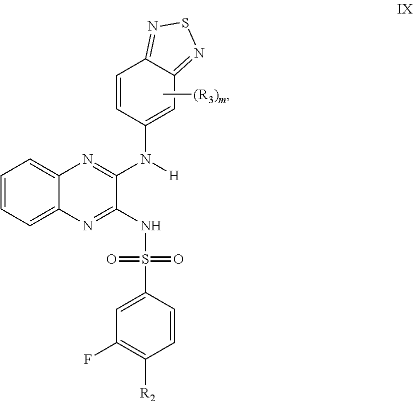
a salt thereof, or a solvate of any of the foregoing;
wherein
R₂ is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamide, ketone, aldehyde, ester, heterocyclyl, aromatic or heteroaromatic moieties, —CF₃, or —CN;
R₃ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamide, ketone, aldehyde, ester, heterocyclyl, aromatic or heteroaromatic moieties, —CF₃, or —CN; and
m is 0, 1, 2, 3, or 4.

3. The compound of claim 1, wherein m is 0.

4. The compound of claim 1, wherein R₂ is chosen from halogen, alkyl, alkoxy, amino, carbonyl, —CF₃, or —CN.

5. The compound of claim 4, wherein R₂ is chosen from methyl, ethyl, propyl, or isopropyl, each optionally substituted with one or more substituents, each independently chosen from F, Cl, Br, hydroxyl, alkoxy, amino, —CF₃, or —CN.

6. The compound of claim 4, wherein R₂ is alkoxy, halogen, amino, or carbonyl.

7-26. (canceled)

27. A compound chosen from: 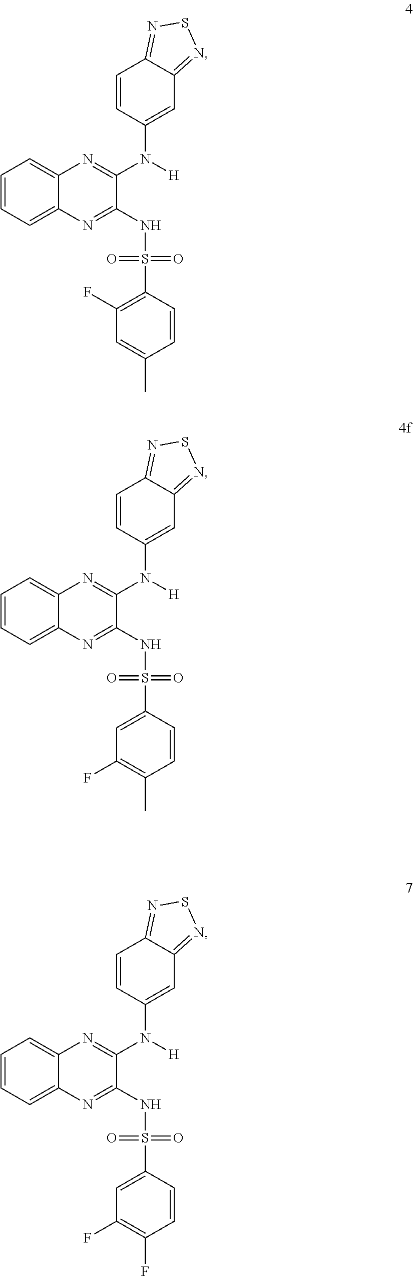 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F ; O=S(=O)(Nc1nc2ccccc2nc1Nc1ccc2nsnc2c1)c1ccc(F)c(F)c1] 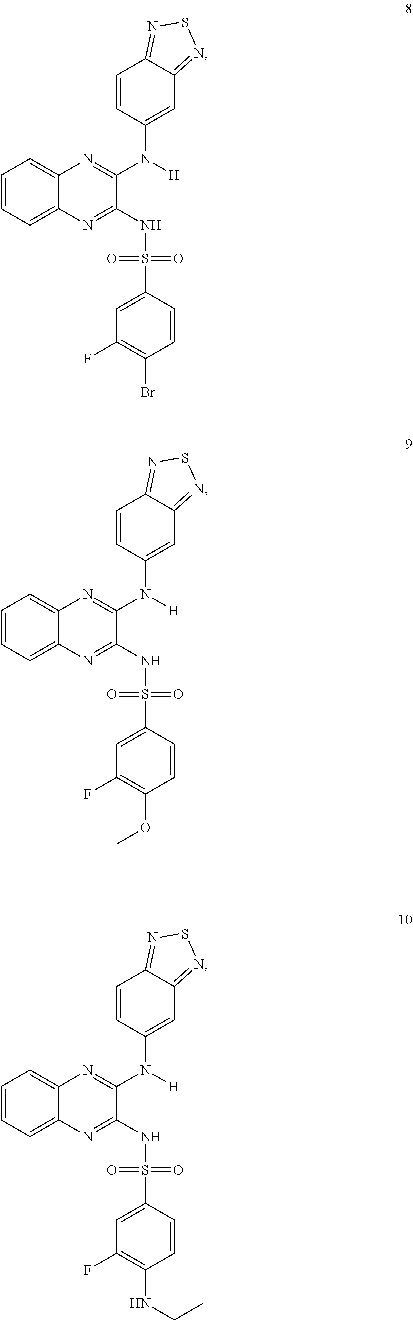 [SMILES: O=S(=O)(Nc1nc2ccccc2nc1Nc1ccc2nsnc2c1)c1ccc(Br)c(F)c1 ; COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F] 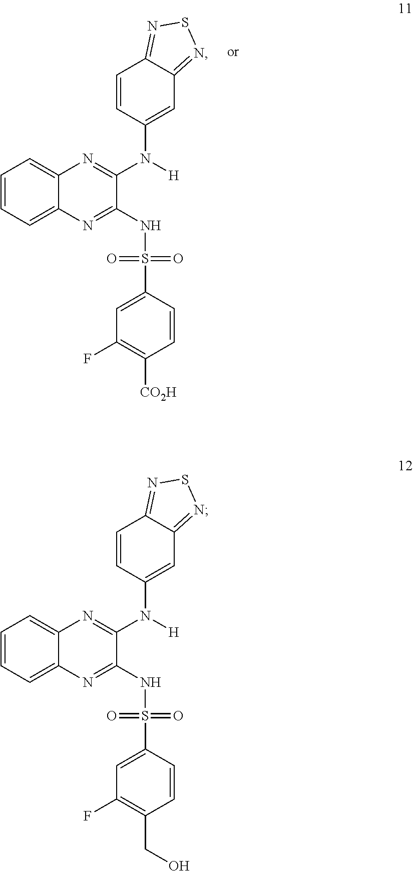 [SMILES: O=C(O)c1ccc(S(=O)(=O)Nc2nc3ccccc3nc2Nc2ccc3nsnc3c2)cc1F ; "or" ; O=S(=O)(Nc1nc2ccccc2nc1Nc1ccc2nsnc2c1)c1ccc(CO)c(F)c1]
a salt thereof, or a solvate of any of the foregoing.

28. The compound of claim 27, wherein the compound comprises Formula 4f: 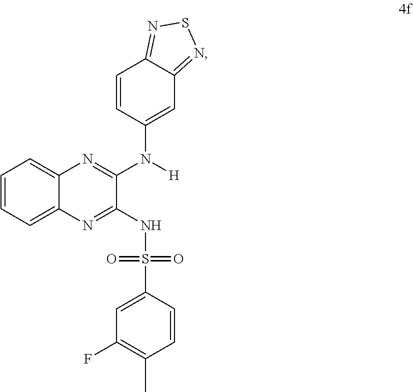 [SMILES: Cc1ccc(S(=O)(=O)Nc2nc3ccccc3nc2Nc2ccc3nsnc3c2)cc1F]
a salt thereof, or a solvate of any of the foregoing.

29. The compound of claim 27, wherein the compound comprises 7: 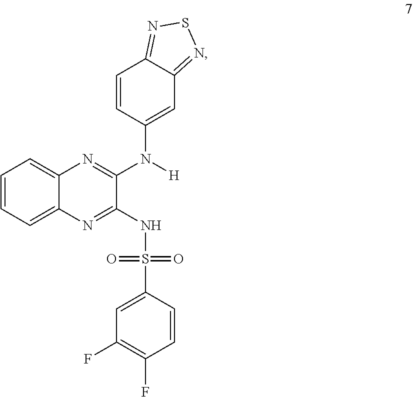 [SMILES: O=S(=O)(Nc1nc2ccccc2nc1Nc1ccc2nsnc2c1)c1ccc(F)c(F)c1]
a salt thereof, or a solvate of any of the foregoing.

30. A composition comprising the compound of claim 37.

31. The composition of claim 30, wherein the composition is a pharmaceutical composition.

32. A method of treating a disease in a subject comprising administering to a subject in need thereof a therapeutically effective amount of at least one compound of claim 37.

33. A method of treating a disease in a subject comprising administering to a subject in need thereof a therapeutically effective amount of at least one compound chosen from: 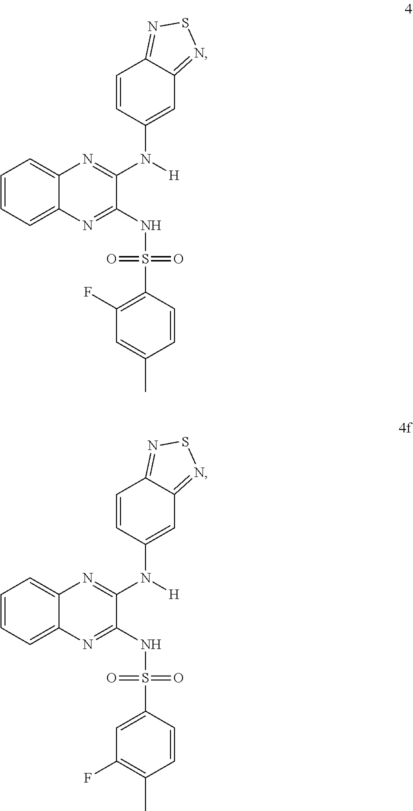 [SMILES: Cc1ccc(S(=O)(=O)Nc2nc3ccccc3nc2Nc2ccc3nsnc3c2)c(F)c1 ; Cc1ccc(S(=O)(=O)Nc2nc3ccccc3nc2Nc2ccc3nsnc3c2)cc1F] 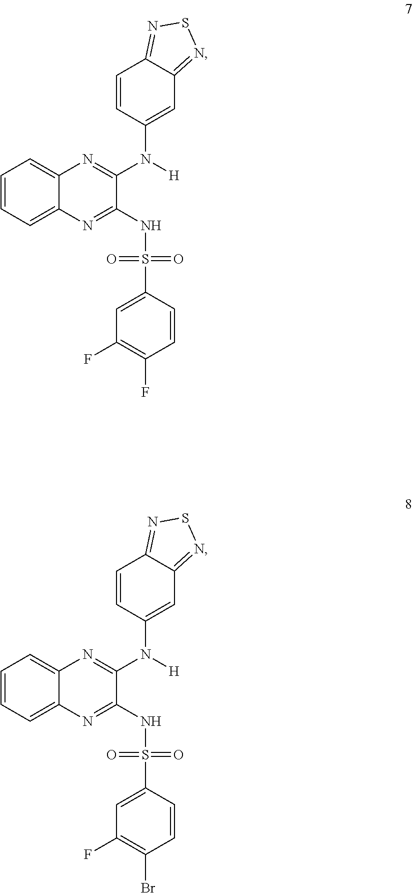 [SMILES: O=S(=O)(Nc1nc2ccccc2nc1Nc1ccc2nsnc2c1)c1ccc(F)c(F)c1 ; O=S(=O)(Nc1nc2ccccc2nc1Nc1ccc2nsnc2c1)c1ccc(Br)c(F)c1] 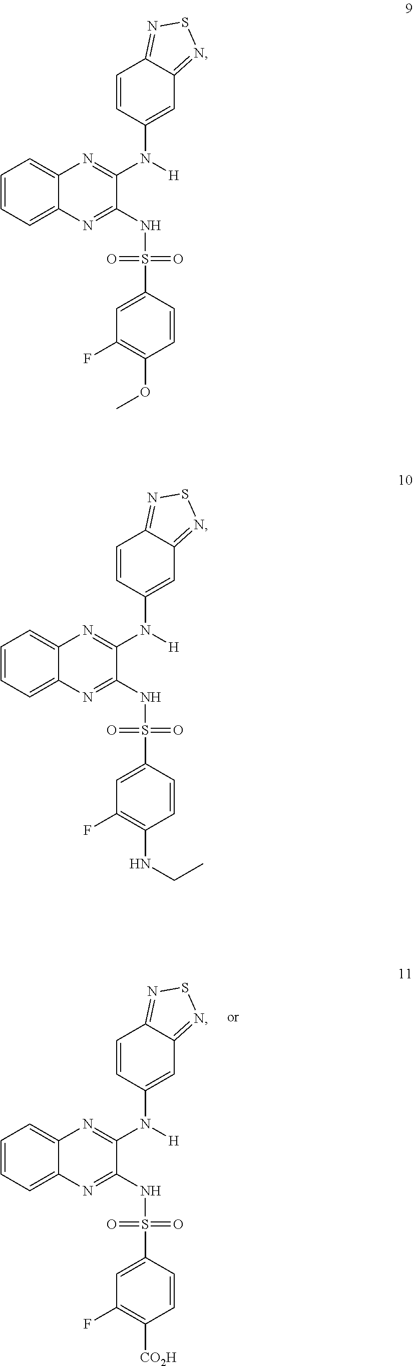 [SMILES: COc1ccc(S(=O)(=O)Nc2nc3ccccc3nc2Nc2ccc3nsnc3c2)cc1F ; CCNc1ccc(S(=O)(=O)Nc2nc3ccccc3nc2Nc2ccc3nsnc3c2)cc1F ; O=C(O)c1ccc(S(=O)(=O)Nc2nc3ccccc3nc2Nc2ccc3nsnc3c2)cc1F ; "or"] 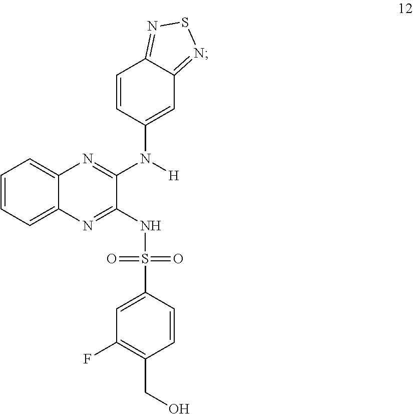 [SMILES: O=S(=O)(Nc1nc2ccccc2nc1Nc1ccc2nsnc2c1)c1ccc(CO)c(F)c1]
a salt thereof, or a solvate of any of the foregoing.

34. The method of claim 33, wherein the disease is cancer.

35. A method of treating a disease in a subject comprising administering to a subject in need thereof a therapeutically effective amount of at least one composition of claim 30.

36. (canceled)

37. A compound of Formula IV: 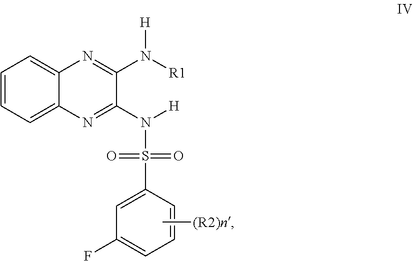
a salt thereof, or a solvate of any of the foregoing;
wherein
R₁ is an aryl group;
R₂ at each occurrence independently is chosen from halogen, azide, alkyl, aralkyl, alkenyl, alkynyl, cycloalkyl, hydroxyl, alkoxy, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, or —CN; and
n′ is 0, 1, 2, 3, or 4.

38. The compound of claim 37, wherein n′ is 0, 1, or 2.

39. The compound of claim 37, wherein the compound has Formula V: [chemical structure]
